(19) United States
(12) Patent Application Publication
(10) Pub. No.: US 2026/0183075 A1
(43) Pub. Date: Jul. 2, 2026

(54) SYSTEMS AND METHODS FOR ROBOTIC ENDOLUMINAL PLATFORM

(71) Applicant: Noah Medical Corporation, San Carlos, CA (US)

(72) Inventors: Enrique ROMO, Danville, CA (US); Matthew Robert PENNY, Holly Springs, NC (US); Daniel NASR-CHURCH, San Diego, CA (US); Craig JOHNSON, Livermore, CA (US); Leobardo Centeno CONTRERAS, East Palo Alto, CA (US); Sizhong ZHANG, San Jose, CA (US); Andrea BAJO, Redwood City, CA (US); Hasan Tutkun SEN, Redwood City, CA (US); Jian ZHANG, Redwood City, CA (US)

(21) Appl. No.: 19/547,964

(22) Filed: Feb. 24, 2026

Related U.S. Application Data
(63) Continuation of application No. PCT/US2024/048599, filed on Sep. 26, 2024.
(60) Provisional application No. 63/586,223, filed on Sep. 28, 2023.

(51) Int. Cl. A61B 34/37; A61B 1/00; A61B 1/015; A61B 1/018; A61B 1/31; A61B 34/30; A61B 90/00; A61M 25/00

(52) U.S. Cl. CPC A61B 34/37; A61B 1/0005; A61B 1/00066; A61B 1/00082; A61B 1/00105; A61B 1/00149; A61B 1/00154; A61B 1/015; A61B 1/018; A61B 1/31; A61B 2034/301; A61B 2090/372; A61B 2562/0223; A61M 2025/0008; A61M 25/005

(57) ABSTRACT

A robotic endoscopic platform is provided. The platform comprises: a robotic endoscopic device comprising a flexible shaft and an articulatable bending section, where the flexible shaft comprises one or more working channels for receiving one or more robotic instruments; a flexible overtube for facilitating an intubation of the robotic endoscopic device into bodily lumen; and a robotic support system comprising a robotic art supporting a robotic end effector, wherein the robotic end effector comprises at least one instrument driving mechanism (IDM) releasably coupled to the robotic endoscopic device and at least one IDM releasably coupled to the one or more robotic instruments.

### CROSS-REFERENCE

This application is a continuation of International Patent Application No. PCT/US2024/048599, filed on Sep. 26, 2024, which claims priority to U.S. Provisional Patent Application No. 63/586,223, filed on Sep. 28, 2023, which is entirely incorporated herein by reference for all purposes.

### BACKGROUND

Endoscopy procedures use an endoscope to examine the interior of a hollow organ or cavity of the body. Unlike many other medical imaging techniques, endoscopes are inserted into the organ directly via the mouth or other naturally occurring orifices. Flexible endoscopes that can deliver instinctive steering and control are useful in diagnosing and treating diseases that are accessible through any natural orifice in the body. Depending on the clinical indication, the endoscope may be designated as colonoscope, gastroscope, bronchoscope, ureteroscope, ENT scope, and various others. For example, a flexible colonoscope may be intubated to transverse colon for diagnosis and/or surgical treatment.

A variety of surgical procedures need to be performed intraluminally, via system introduction through natural orifices in the gastrointestinal tract. Broadly, these surgical procedures may include tissue removal, tissue repair and tissue restructuring. More specifically, the procedures may include, but are not limited to, endoscopic submucosal dissection (ESD), endoscopic mucosal resection (EMR), Endoscopic Gastric Plication, full thickness resection and the like. Current tools limit the capabilities and the procedural applicability for clinicians operating endoluminal interventions and complex surgical procedures. For example, clinicians performing endoscopic submucosal dissection in the colon have to “build” their own system for access, dissection and closure from available equipment such as colonoscopes, cutters and over-the-scope closure technologies. Additionally, clinicians may not be able to simultaneously control all aspects of the surgical procedure. For example, the clinician is in control of the manual colonoscope throughout the procedure, but can only control the insertion of instruments through the auxiliary channel. The surgical support staff is often responsible for fluid injection (to lift lesions from the colon wall), rotational orientation of electrocautery tools such as “L” hooks and open/closure tools such as biopsy forceps or polyp snares. This requires substantial verbal communication and coordination throughout the intervention which introduces variability and error.

### SUMMARY

Recognized herein is a need for a robust and instinctive system for performing complex surgical procedures. The present disclosure provides a robust and instinctive system for performing complex surgical procedures particularly through natural orifices in the gastrointestinal tract.

In an aspect, a robotic endoscopic platform is provided. The robotic endoscopic platform comprises: a robotic endoscopic device comprising a flexible shaft and an articulatable bending section, wherein the flexible shaft comprises one or more working channels for receiving one or more robotic instruments; a flexible overtube for facilitating an intubation of the robotic endoscopic device into a lumen; a robotic support system comprising a robotic arm supporting a robotic end effector, wherein the robotic end effector comprises at least one instrument driving mechanism (IDM) releasably coupled to the robotic endoscopic device and at least one IDM releasably coupled to the one or more robotic instruments; and a user console comprising a user input device for controlling an operation of the one or more robotic instruments and a viewing apparatus for accessing a real-time camera view with one or more overlay elements.

In some embodiments, a distal end of the robotic endoscopic device is steered by the articulatable bending section and wherein the distal end is embedded with at least an imaging device and a light source. In some embodiments, the articulatable bending section is controlled by one or more pull wires.

In some embodiments, the flexible overtube comprises a layflat tube construction. In some cases, the flexible overtube comprises a first lumen for receiving the robotic endoscopic platform and a second lumen for receiving a manual scope.

In some embodiments, the one or more robotic instruments comprise a needle delivery tool. In some cases, a needle loaded to the needle delivery tool is grasped by a grasper instrument that is inserted through the working channel of the one or more working channels.

In some embodiments, the user input device comprises a shoulder assembly, a wrist assembly and a pincher assembly for capturing a user action and converting the user action to a control signal for controlling an operation of an end effector of the one or more robotic instruments and a position and orientation of the end effector. In some cases, the user input device comprises a passive compensation mechanism. In some cases, the pincher assembly comprises a clutch switch for repositioning the wrist assembly.

In some embodiments, the one or more overlay elements may comprise a graphical indicator corresponding to a foot pedal. In some cases, the graphical indicator is displayed to indicate presence of a foot hovering over the foot pedal. In some instances, the presence of the foot is detected by a hover sensor.

In some embodiments, the viewing apparatus is a stereo viewing device. In some cases, the viewing apparatus is adjustable to permit an upright viewing posture. In some cases, a plurality of the foot pedals are arranged on a tiered foot pedal tray.

In another aspect, a method for intubating a robotic endoscope into a subject is provided. The method comprises: performing an initial intubation to reach a target site within a body of the subject with a first scope and an overtube, wherein the first scope is a manual scope having a first diameter; withdrawing the first scope and inserting the robotic endoscope through the overtube until a tip of the robotic endoscope reaches a distal end of the overtube; and deflating a balloon of the overtube and exposing a bending section of the robotic endoscope by retracting the overtube.

In some embodiments, the method further comprises coupling a dilator to the overtube and the first scope such that the dilator occupies a space between the overtube and the first scope. In some embodiments, the first diameter of the first scope is smaller than a diameter of the robotic endoscope. In some embodiments, the robotic endoscope is a gastroscope or colonoscope.

It should be noted that the provided suturing device, end effector, endoscope components and various components of the device can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others. The devices and systems can be used in any subject that may or may not involve human body, animal, or tissue.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically shows an example of a flexible robotic endoscope, in accordance with some embodiments of the present disclosure.

FIG. 2 shows an example of a handle portion of a flexible robotic endoscope, in accordance with some embodiments of the present disclosure.

FIG. 3 shows an example of a distal tip of a flexible robotic endoscope.

FIG. 4 shows an example of a robotic support system with instrument driving mechanism.

FIG. 5 shows examples of instrument driving mechanism at a robotic end effector.

FIG. 6 shows an example of a robotic arm mounted to a robotic mount base.

FIG. 7 shows examples of instrument driving mechanism for driving robotic endoscope and robotic instruments at a robotic end effector.

FIG. 8 shows examples of instrument driving mechanism for driving robotic instruments at a robotic end effector.

FIG. 9 shows an example of a treatment control system or a robotic cart.

FIG. 10 shows an example of an overtube used for reducting an anatomy and facilitating scope exchange.

FIG. 11 illustrates an overtube device having a layflat tube construction.

FIGS. 12A-12B shows an example of a dilator/obturator used in conjunction with an overtube.

FIG. 13A shows an example of an overtube device with reinforcement features.

FIG. 13B shows an exemplary intubation workflow for an endoscope device.

FIG. 14 schematically shows an example of a robotic instrument.

FIG. 15 shows an example of performing an endoscopic submucosal dissection (ESD).

FIGS. 16-20 show various examples of end effectors for robotic instruments.

FIG. 21 shows an example of a user console.

FIG. 22 shows examples of user input devices.

FIG. 23 shows examples of the user console with adjustable viewing apparatus.

FIG. 24 shows an example of a stereo viewer apparatus.

FIG. 25 shows an example of seven joints (J1-J7) of each arm actuated by seven motors.

FIG. 26 shows an example of a pincher assembly and various components of the pincher assembly.

FIGS. 27 and 28 show examples of a passive compensation feature of the user input device.

FIGS. 29 and 30 show examples of the display of the stereo viewer apparatus that includes a live camera view with overlay elements.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Recognized herein is a need for a minimally invasive system that allows for performing surgical procedures or diagnostic operations with improved reliability and cost-efficiency. The present disclosure provides a robust and instinctive system for performing complex surgical procedures particularly through natural orifices in the gastrointestinal tract.

In some embodiments, the present disclosure provides an Endoluminal Surgical Platform comprising a robotic system for natural orifice intervention of lower and upper gastroenterology (GI) clinical conditions that can be reached and addressed intraluminally. In some embodiments, the robotic platform may comprise a robotically manipulated endoscope i.e., a colonoscope for lower GI, and/or gastroscope for upper GI. The Endoluminal Surgical Platform herein may comprise devices and systems for gaining access to the endoluminal site of interest, devices and systems of introducing and placing the primary robotic endoscope, and devices and systems of introducing and exchanging secondary robotic instruments through a primary endoscope.

The Endoluminal Surgical Platform may have capabilities to visualize and deliver the necessary diagnostic and therapeutic tasks, tools and systems for retrieving excised tissue from the patient, tools and systems of enabling tissue approximation and closure, and structures and features for managing fluidics, insufflation, and smoke management intraoperatively. The robotic system and platform of the present disclosure may allow for complex surgical procedures including tissue removal, tissue repair and tissue restructuring and the like to be performed with improved reliability and performance. Examples of surgical procedures may include, but are not limited to, endoscopic submucosal dissection (ESD), endoscopic mucosal resection (EMR), Endoscopic Gastric Plication, full thickness resection and the like.

While exemplary embodiments will be primarily directed at a device or system for colonoscope or gastroscope, one of skill in the art will appreciate that this is not intended to be limiting, and the devices described herein may be used for other therapeutic or diagnostic procedures and in various anatomical regions of a patient's body. The provided device or system can be utilized in urology, gynecology, rhinology, otology, laryngoscopy, gastroenterology with the endoscopes, combined devices including endoscope and instruments, endoscopes with localization functions, one of skill in the art will appreciate that this is not intended to be limiting, and the devices described herein may be used for other therapeutic or diagnostic procedures and in other anatomical regions of a patient's body, such as such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat, and various others, in the forms of: NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, Orthopedic Endoscopes, and others, in combination with various tools or instruments.

The embodiments disclosed herein can be combined in one or more of many ways to provide improved diagnosis, therapy or surgical operations to a patient. It is to be understood that any one or more of the structures and steps as described herein can be combined with any one or more additional structures and steps of the methods and apparatus as described herein, the drawings and supporting text provide descriptions in accordance with embodiments.

Although the treatment planning and definition of diagnosis or surgical procedures as described herein are presented in the context of diagnosis or surgery in lower and upper gastroenterology clinical conditions, the methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) or a microcontroller), digital signal processors (DSPs), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus, and can be opposite of anatomical references. For example, a distal location of an endoscope or catheter may correspond to a proximal location of an elongate member of the patient, and a proximal location of the endoscope or catheter may correspond to a distal location of the elongate member of the patient.

A system as described herein, includes an elongate portion or elongate member such as a catheter. The terms “elongate member”, and “catheter” are used interchangeably throughout the specification unless contexts suggest otherwise. The terms “endoscope,” “scope,” “gastroscope,” and “colonoscope” are used interchangeably throughout the specification unless contexts suggest otherwise. The elongate member can be placed directly into the body lumen or a body cavity through natural orifice. In some embodiments, the system may further include a support apparatus such as a robotic manipulator (e.g., robotic arm) to drive, support, position or control the movements and/or operation of the elongate member. Alternatively or in addition to, the support apparatus may be a hand-held device or other control devices that may or may not include a robotic system. In some embodiments, the system may further include peripheral devices and subsystems such as imaging systems that would assist and/or facilitate the navigation of the elongate member to the target site in the body of a subject.

### Robotic Scope

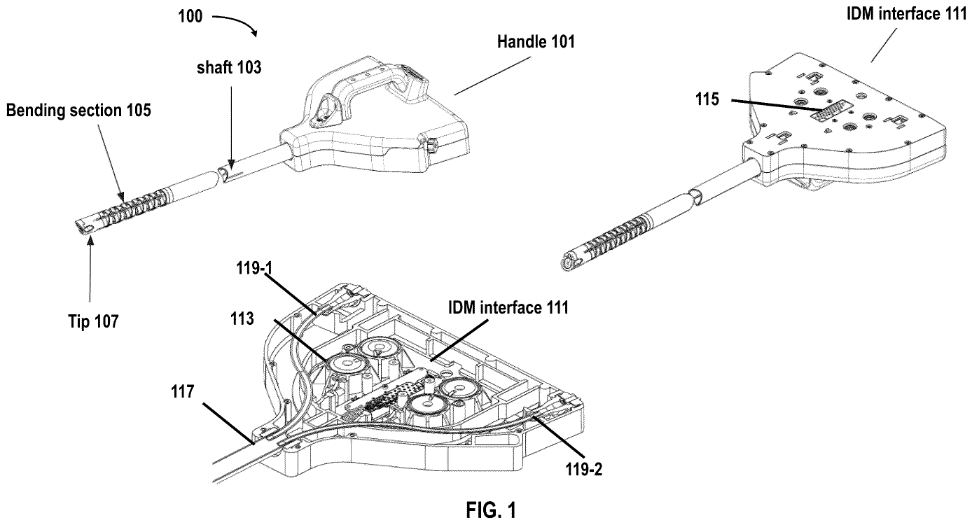

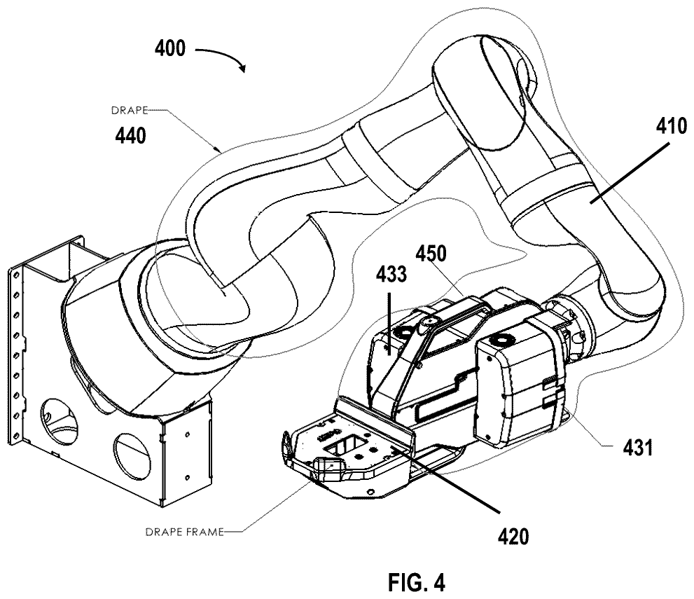

The Endoluminal Surgical Platform or the robotic scope system herein may comprise a robotic colonoscope/gastroscope (e.g., steerable catheter assembly) and a robotic support system, for supporting or carrying the robotic colonoscope. The steerable catheter assembly can be a colonoscope. FIG. 1 shows an example of a robotic colonoscope or steerable catheter assembly 100. In some embodiments, the steerable catheter assembly may be a single-use robotic colonoscope/gastroscope. In some embodiments, the robotic scope system may comprise an instrument driving mechanism (e.g., IDM 420 in FIG. 4) that is attached to the arm of the robotic support system (e.g., robotic arm 410 in FIG. 4) and the steerable catheter assembly may be releasably attached to the IDM. The instrument driving mechanism (IDM) may be provided by a suitable controller device that includes a robotic system. Alternatively, the instrument driving mechanism may be provided by any suitable controller device (e.g., hand-held controller) that may not include a robotic system. The instrument driving mechanism may provide mechanical and electrical interface to the steerable catheter assembly 100. The mechanical interface may allow the steerable catheter assembly 100 to be releasably coupled to the instrument driving mechanism. For instance, a handle portion 101 of the steerable catheter assembly can be attached to the instrument driving mechanism via quick install/release means, such as magnets, spring-loaded levers and the like. In some cases, the steerable catheter assembly may be coupled to or released from the instrument driving mechanism manually without using a tool.

The steerable catheter assembly 100 may comprise a handle portion 101 that may include components configured to process image data, provide power, or establish communication with other external devices. For instance, the handle portion 100 may include a circuitry and communication elements that enables electrical communication between the steerable catheter assembly 100 and the instrument driving mechanism, and any other external system or devices. In another example, the handle portion 101 may comprise circuitry elements such as power sources for powering the electronics (e.g., camera and LED lights) of the endoscope. In some cases, the handle portion may be in electrical communication with the instrument driving mechanism via an electrical interface (e.g., printed circuit board) so that image/video data and/or sensor data can be received by the communication module of the instrument driving mechanism and may be transmitted to other external devices/systems. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

As shown in FIG. 1, the flexible robotic endoscope 100 may comprise a handle/proximal portion 101 and a flexible elongate member to be inserted inside of a subject. The flexible elongate member may comprise at least a distal tip portion 107, a bending section 105 and a shaft 103. In some cases, the flexible robotic endoscope 100 may also be referred to as steerable catheter assembly as described elsewhere herein. In some cases, the flexible robotic endoscope may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiments, the endoscope may contain varying levels of stiffness along the shaft, as to improve functional operation.

The endoscope or steerable catheter assembly 100 may comprise a handle portion 101 that may include one or more components configured to process image data, provide power, or establish communication with other external devices. For instance, the handle portion may include a circuitry and communication elements 115 that enables electrical communication between the steerable catheter assembly and an instrument driving mechanism (e.g., IDM 420 in FIG. 4), and any other external system or devices. In another example, the handle portion 101 may comprise circuitry elements such as power sources for powering the electronics (e.g., camera, electromagnetic sensor and LED lights) of the endoscope. The one or more components located at the handle may be optimized such that expensive and complicated components may be allocated to the robotic support system (e.g., 400 in FIG. 4), a hand-held controller or an instrument driving mechanism (e.g., IDM 420 in FIG. 4) thereby reducing the cost and simplifying the design of the disposable endoscope.

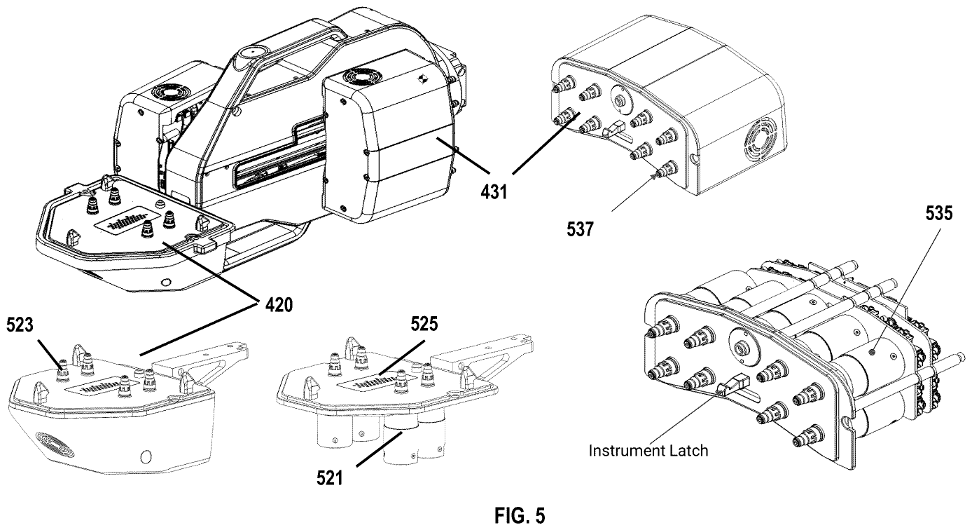

The handle portion or proximal portion 101 may provide an electrical interface 115 and mechanical interface 113 to allow for electrical communication and mechanical communication with the instrument driving mechanism (e.g., IDM 420 in FIGS. 4 and 5). As shown in FIG. 5, the instrument driving mechanism 420 for controlling the endoscope may comprise a set of motors 521 that are actuated to rotationally drive a set of pull wires 117 of the catheter. The handle portion of the catheter assembly may be mounted onto the instrument drive mechanism so that its pulley/capstans assemblies 113 are driven by the set of motors 521 via the output shaft 523. In some embodiments, instead of or in addition to a rotary interface, the IDM may have a linear interface. The number of pulleys may vary based on the pull wire configurations. In some cases, one, two, three, four, or more pull wires may be utilized for articulating the flexible endoscope or catheter.

The handle portion 101 may be designed allowing the flexible robotic endoscope to be disposable at reduced cost. For instance, classic manual and robotic endoscopes may have a cable in the proximal end of the endoscope handle. The cable may comprise illumination fibers, camera video cable and the like. In some cases, the cables may comprise other sensors fibers or cables such as electromagnetic (EM) sensors, or shape sensing fibers. Such complex cable can be expensive adding to the cost of the endoscope. The provided flexible robotic endoscope may have an optimized design such that simplified structures and components can be employed while preserving the mechanical and electrical functionalities. In some cases, the handle portion of the robotic endoscope may employ a cable-free design while providing a mechanical/electrical interface to the catheter as described later herein.

The electrical interface (e.g., printed circuit board 115) may allow image/video data and/or sensor data to be received by the communication module of the instrument driving mechanism (e.g., IDM 420 in FIG. 4) and may be transmitted to other external devices/systems. In some cases, the electrical interface may establish electrical communication without cables or wires. For example, the interface may comprise pins soldered onto an electronics board such as a printed circuit board (PCB) 115. For instance, a receptacle connector (e.g., the female connector) 525 is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

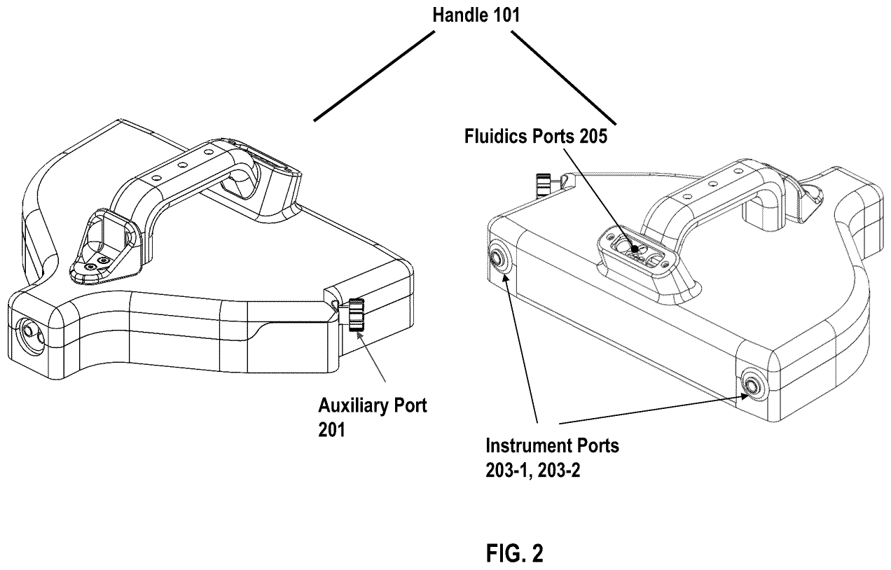

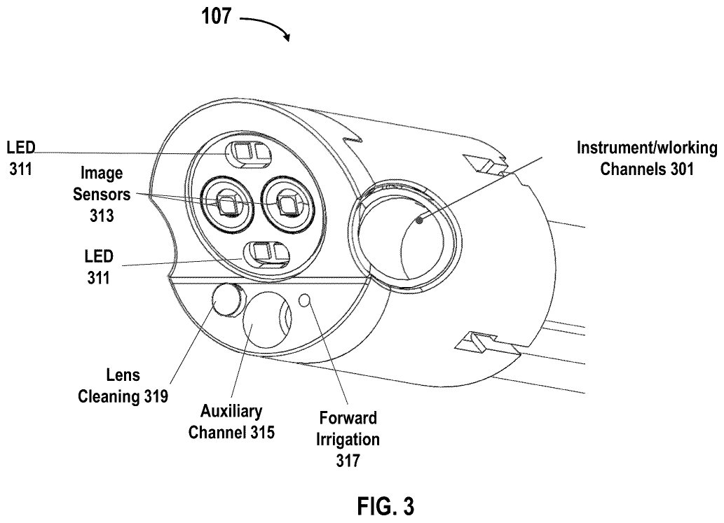

In some cases, the handle portion 101 may comprise one or more mechanical control modules for controlling irrigation system/aspiration system, or mechanical structures 191-1, 191-2 for guiding or aligning one or more working channels with an inserting trajectory of the robotic endoscope FIG. 2 shows an example of the handle portion. The handle portion can be the same as those described above. For example, the handle portion may comprise one or more mechanical control modules such as luer, or fluidics ports 205 for interfacing the irrigation system/aspiration system. The robotic endoscope may comprise fluidics channels for insufflation (e.g., CO₂), camera rinse, forward irrigation and/or smoke evacuation. For example, Carbon dioxide (CO2) insufflation may be provided using of CO2 gas to inflate a space, such as the abdomen or digestive tract, for a variety of medical procedures. FIG. 3 shows an example of a distal tip of the robotic endoscope including a forward irrigation fluidics channel 317 and a lens cleaning fluidics channel 319. Irrigation system may be coupled to the fluids channels via the one or more fluidics ports 205. In some cases, such insufflation and smoke exhaust functionalities or the fluidics channels may be located in the clearance between the endoscope shaft(s) and working channel(s). In some cases, the robotic endoscope may comprise one or more nozzles for clearing a camera view. For instance, the distal tip may comprise one or more irrigation ports such as a forward irrigation nozzle 317 and a window cleaning nozzle for providing a clear camera view. For example, an irrigation and aspiration system may connect to the fluidics channels and/or the working channel for the robotic endoscope through the fluidics ports 205. The irrigation system can inject fluids such as saline and the aspiration system may aspire mucus or saline or other material out of the airways. In some cases, the fluidics channels may be the same as the instrument channels, where the fluidics functions may be operable with or without an instrument present. For instance, fluidics (e.g., insufflation, forward irrigation or suction) may be performed through an instruments channel.

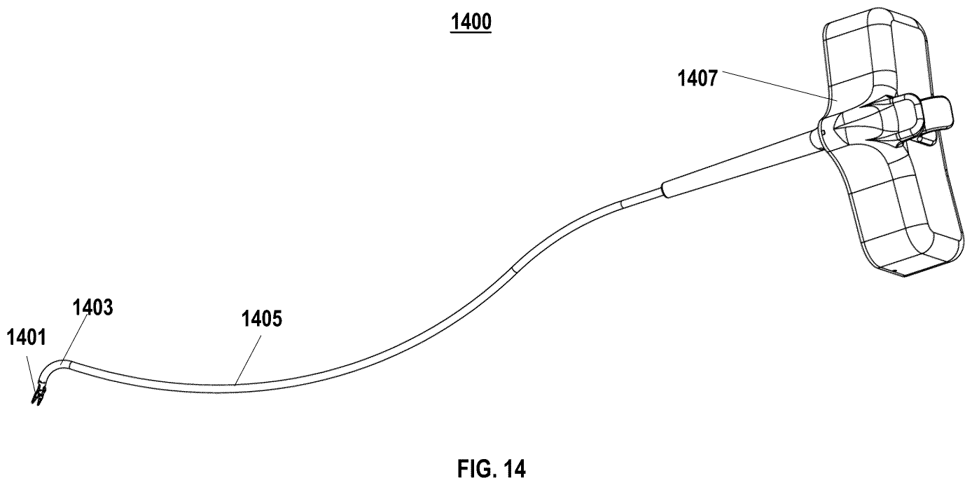

The handle portion 101 may further comprise mechanical structures 191-1, 191-2 such as a working channel guidance system for aligning one or more of the working channels of the robotic colonoscope/gastroscope with an insertion trajectory of one or more robotic instruments when both the colonoscope/gastroscope and the robotic instruments are coupled to the robotic IDMs. The robotic colonoscope/gastroscope may comprise one or more working channels or instrument channels 301. The working channel (e.g., working channels or instrument channels 301) may be designed to provide protection for the internal components such as flexible robotic instruments (e.g., suturing instrument, forceps, etc.). When flexible robotic instruments pass through a conventional working channel, they may be obstructed by the working channel due to kinking, ovalizing and/or high friction force. The working channel herein may provide a high hoop strength and a capability of achieving low bend radius. The working channel may also be designed to provide low friction in the inner surface. One or more robotic instrument (e.g., suturing instrument, cutter, forceps, grasper, needle delivery tool and the like as described with respect to FIGS. 14-20) may be passed through the working channel and advanced over the distal tip of the endoscope or retracted back into the working channel. The various robotic instruments may be independently steerable from the robotic endoscope. Details about the robotic instruments are described later herein.

As shown in FIG. 2, the handle portion may comprise one or more instrument ports 203-1, 203-2 for receiving the robotic instrument. In some embodiments, the robotic endoscope may comprise at least one working channel(s) for the robotic instruments. In some embodiments, any of the one or more working channels may also be used for fluidic transport. The handle portion may comprise working channel seals for minimizing fluid egress.

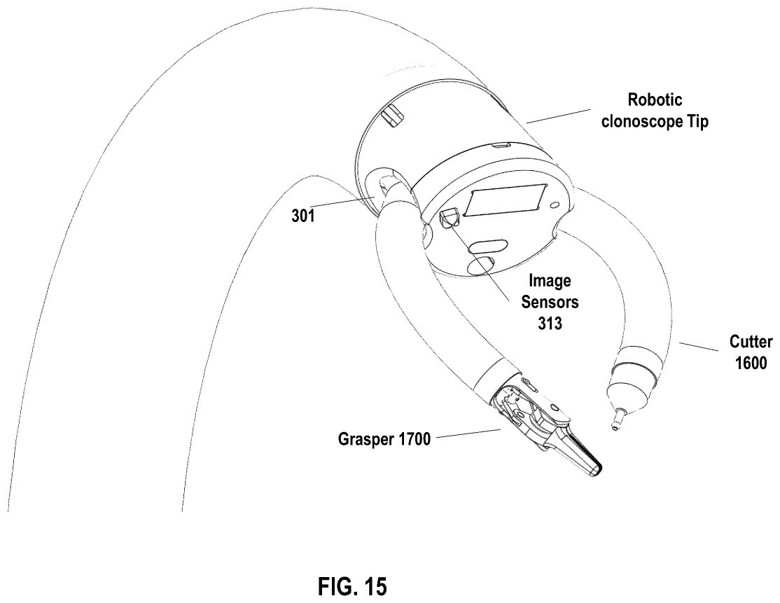

As shown in FIG. 3, in some embodiments, the one or more working channels 301 may exit the distal tip 107 of the robotic endoscope in a direction that diverges from a primary axis of the robotic endoscope tip. The exit or the exit port of the working channel may be located at the distal portion allowing the robotic instruments to have triangulation by making the arms spread out or divert away from the base as they exit the distal portion of the primary sheath. FIG. 15 shows an example of robotic instruments extended through the exit ports of the working channels to perform endoluminal operations. As shown in the example, the exit ports may allow the robotic instruments to have triangulation by making the arms spread out or divert away from the base as they exit the distal portion of the primary sheath distal portions and then be steered back toward each other and utilized to apply capturing and/or compressive loads to a subject tissue structure, and the like, with the field of view of the image capture device 313 preferably capturing such activity from any desired location relative to the robotic instruments (e.g., grasper, cutter). In some embodiments, a location of the exit port of the working channel may be substantially at a circumferential side of the distal tip closer to the front end. In some cases, the location of the exit port may be located at the edge where a side and a front end of the distal tip meets. As shown in FIG. 15, the two instrument exit ports may be located on substantially opposite sides of the port for the endoscope. This configuration may allow for surgical triangulation with the distal portions of the robotic instrument assemblies (e.g., grasper 1700, cutter 1600).

In some embodiments, the robotic colonoscope/gastroscope may comprise at least one auxiliary channel 315 for receiving a non-robotic instrument. A non-robotic instrument may be any flexible instrument without robotic control features. The non-robotic instrument may be inserted through the auxiliary channel such as for delivering material or tools to the distal tip or perform other function without the need to be robotically controlled. The handle portion may comprise an auxiliary channel port 201 for inserting the non-robotic instrument. In some cases, the auxiliary channel can be configurable for either non-robotic instrument or as an additional fluid delivery. For example, the auxiliary channel port 201 may be connected to an irrigation system when additional fluidics is desired.

In the illustrated example, the distal tip 107 of the robotic endoscope is configured to be articulated/bent in two or more degrees of freedom to provide a desired camera view or control the direction of the endoscope. As illustrated in FIG. 3, imaging device 313 (e.g., camera), position sensors (e.g., electromagnetic sensor) may be embedded in the distal tip of the catheter or endoscope shaft. For example, line of sight of the camera may be controlled by controlling the articulation of the active bending section 105. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with the aid of an optical component.

The distal tip 107 may be a rigid component that allows for imaging devices (e.g., camera) and other electronic components 311 (e.g., LED light source) being embedded at the distal tip. Depending on the type of the endoscope, the distal tip may comprise other sensors such as electromagnetic (EM) sensors or inertial measurement units embedded in the distal tip.

The robotic endoscope may or may not have real-time EM tracking capability. In some embodiments, the robotic endoscope may not have a positional sensor (electromagnetic sensor) for tracking a location of the distal tip of the endoscope during navigation while the live camera view may allow an operator to identify a location of the endoscope tip via visual feedback. Alternatively, when the robotic endoscope is embedded with EM sensor, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient during a procedure to locate the EM sensor position in 3D space or may locate the EM sensor position and orientation in 5D or 6D space. This may provide a visual guide to an operator when driving the endoscope towards the target site.

The robotic endoscope may have a dimension so that one or more electronic components can be integrated to the endoscope. For example, as shown in FIG. 3, the outer diameter of the distal tip 107 may range from 3 mm to 25 mm, and the diameter of the instrument channels 301 may range from 2 mm to 8 mm such that one or more instruments can be removably inserted through the endoscope to the surgical site. However, it should be noted that based on different applications, the outer diameter can be in any range smaller than 3 mm or greater than 25 mm, and the diameter of the instrument channels 301 can be in any range such as about 4 mm or 5 mm to allow the robotic instrument herein passing through. The space not occupied by fluidics or instrument pass throughs can be used to embed electronic components into the wall of the endoscope.

Due to the offset arrangement of the instrument channel (i.e., not along the central axis of the shaft), if a tension force is exerted to the distal tip of an instrument that is inserted through the instrument channel (e.g., needle retraction cable is attempting to maintain a tension to keep the needle retracted), the instrument tip may be biased up against the distal end of the bending section (e.g., tend to bend the bending section up). To ensure the instrument tip stays coupled to the distal end of the bending section while the length of the instrument flexible shaft in the bending section can change, a force exerted to the instrument tip (e.g., tension in the needle retraction cable) may be monitored to keep the force (e.g., tension in cable) at a target range. For example, in the case of a needle instrument, a retraction cable is maintaining a tension, the motor controlling needle retraction may rotate to maintain tension. The target tension range may be maintained by providing wire slack if tension is too high, or removing wire slack if the tension is too low. In some cases, the maintenance of the target tension may provide an estimation of the bending degree, or bending orientation of the bending section. For example, an amount of angular rotation on the motor for driving the needle retraction cable may be correlated to the bending angle (direction and degree) of the bending section. In some cases, the bending section orientation or bending angle may be estimated based on the robotic control signal for articulating the bending section. In some cases, the bending section orientation or bending angle may be estimated based on motor current and angle change of the motor for driving a pull wire attached to the bending section.

The one or more electronic components may comprise an imaging device, illumination device or other optional sensors. In some embodiments, the imaging device may be a video camera 313. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 311 positioned at the distal tip. The light source may be a light-emitting diode (LED), an organic LED (OLED), a quantum dot, or any other suitable light source. In some cases, the light source may be a miniaturized LED for a compact design or Dual Tone Flash LED Lighting.

The imaging device and the illumination device may be integrated to the endoscope. For example, the distal portion of the endoscope may comprise suitable structures matching at least a dimension of the imaging device and the illumination device. The imaging device and the illumination device may be embedded into the catheter. A camera may be located at the distal portion 107. The distal tip may have a structure to receive the camera, and illumination device. For example, the camera may be embedded into a cavity at the distal tip of the catheter. The cavity may be integrally formed with the distal portion of the cavity and may have a dimension matching a length/width of the camera such that the camera may not move relative to the endoscope. The camera may be adjacent to one or more instrument channels 301 of the endoscope to provide near field view of the tissue or the organs. In some cases, the viewing direction or orientation of the imaging device may be controlled by controlling a rotational movement (e.g., roll) of the endoscope.

The power to the camera may be provided by a wired cable. In some cases, the cable wire may be in a wire bundle providing power to the camera as well as illumination elements or other circuitry at the distal tip of the robotic endoscope. The camera and/or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light 311 may be provided by fiber cables that transfer the light of a light source located at the proximal end of the endoscope, to the distal end of the robotic endoscope. In some embodiments of the disclosure, miniaturized LED lights may be employed and embedded into the distal portion of the catheter to reduce the design complexity. In some cases, the distal portion may comprise a structure having a dimension matching a dimension of the miniaturized LED light source. As shown in the illustrated example, two cavities may be integrally formed with the endoscope to receive two LED light sources 311. For instance, the outer diameter of the distal tip may range from 3 mm to 25 mm and diameter of the working channel of the endoscope may be around 4.5 or 6 mm such that two LED light sources may be embedded at the distal end. The outer diameter can be in any range smaller than 3 mm or greater than 25 mm, and the diameter of the instrument channels 301 can be in any range according to the tool's dimensional or specific application. Any number of light sources may be included. The internal structure of the distal portion may be designed to fit any number of light sources.

In some cases, each of the LEDs may be connected to power wires which may run to the proximal handle 101. In some embodiments, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover may be placed at the forwarding end face of the distal tip providing precise positioning of the LEDs as well as sufficient room for the glue. The cover may be composed of transparent material matching the refractive index of the glue so that the illumination light may not be obstructed.

The robotic endoscope may have a unique design in the elongate member. In some cases, the active bending section 105 and the shaft 103 of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius). In some cases, the shaft 103 may comprise a composite structure including coils, braids and polymers for constructing the shaft to meet a desired stiffness and compliance attributes.

As described above, the active bending section 105 may be designed to allow for bending in two or more degrees of freedom (e.g., articulation). A greater bending degree such as 180 and 270 degrees (or other articulation parameters for clinical indications) can be achieved by the unique structure of the active bending section. In some cases, the active bending section may be fabricated separately as a modular component and assembled to the shaft. In some cases, the active bending section may comprise features or a construction at selected locations such that at least a minimum bend radius of the bending section may vary along the length. In some cases, a variable minimum bend radius along the axial axis of the elongate member may be provided. In some cases, features, such as reliefs, maybe formed into the active bending section via a wide variety of manufacturing techniques including, but not limited to, laser cutting, injection molding, casting, forming and other means that are known to those skilled in the art to vary the minimum bend radius, or stiffness. The construction of the active bending section may be achieved through various manufacturing methods such as additive manufacturing, injection molding, laser cutting or other techniques.

The articulation of the endoscope may be controlled by applying force to the distal end of the endoscope via one or multiple pull wires 117. The one or more pull wires may be attached to the distal end of the endoscope (e.g., at the distal end of the bending section 105 or the proximal end of the tip 107). In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle where they are coupled to a driving component (e.g., pulley). This handle pulley 113 may interact with an output shaft 523 from the robotic system.

In some embodiments, the proximal end or portion of one or more pull wires 117 may be operatively coupled to various mechanisms (e.g., gears, pulleys, capstans, etc.) 113 in the handle portion of the catheter assembly. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g., SS316), metals, alloys, polymers, nylons or biocompatible material. Pull wires may be a wire, cable or a thread. In some embodiments, different pull wires may be made of different materials for varying the load bearing capabilities of the pull wires. In some embodiments, different sections of the pull wires may be made of different material to vary the stiffness and/or load bearing along the pull. In some embodiments, pull wires may be utilized for the transfer of electrical signals.

In some embodiments, the provided robotic endoscope can be a single-use endoscope that may beneficially reduce cross-contamination between patients and infections. In some cases, the robotic gastroscope may be delivered to the medical practitioner in a pre-sterilized package and are intended to be disposed of after a single use. The proximal design may improve the reliability of the device without introducing extra cost allowing for a low-cost single-use endoscope. Alternatively, the robotic endoscope may be reusable.

The robotic endoscope 100 may be attached to a robotic support system 400 via the instrument driving mechanism 420 as shown in FIG. 4. Alternatively, the robotic endoscope may be attached to a hand-held controller via an IDM on the hand-held controller. The instrument driving mechanism (IDM) may be provided by any suitable controller device (e.g., hand-held controller) that may or may not include a robotic system. The instrument driving mechanism may provide mechanical and electrical interface to the steerable catheter assembly 100. The mechanical interface may allow the steerable catheter assembly 100 to be releasably coupled to the instrument driving mechanism as described elsewhere herein.

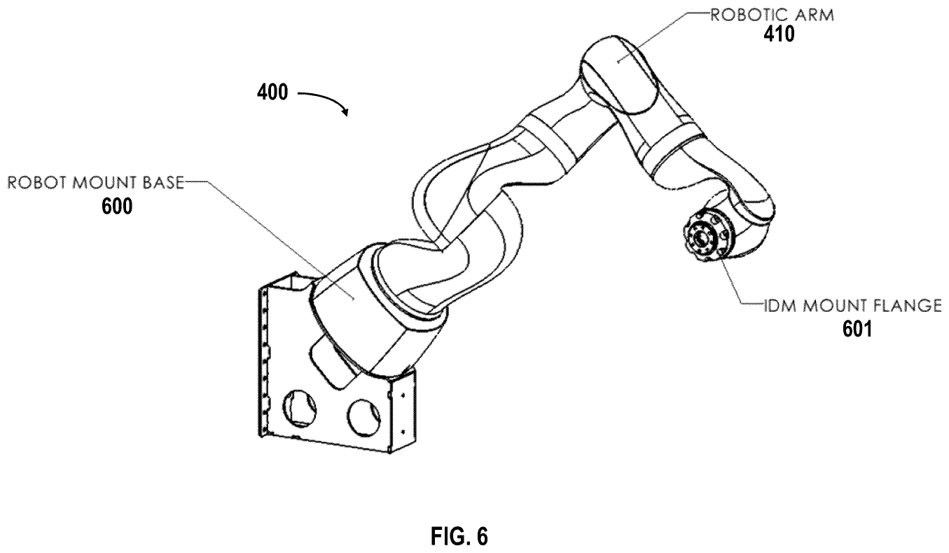

In some embodiments, the robotic support system 400 may support one or more IDMs 431, 433 for driving operations of one or more robotic instruments and an IDM 420 for driving operations of the robotic endoscope. In some embodiments, the robotic support system 400 may comprise a robotic arm 410 supporting the one or more IDMs as an end effector. FIGS. 4, 6 and 9 shows an example of a robotic arm 410 mounted on top of a robot cart 900 (e.g., a bed-side cart in a treatment control system) via a robot mount base 600. The robotic arm may automatically position the end effector such as the one or more IDMs to any desired position. The end effector of the robotic arm such as the one or more IDMs may have at least three degrees of freedom (e.g., x, y, z translational movement). In some cases, the one or more IDMs may also have pitch, yaw, or roll movement with respect to the robot mount base 600. For example, the roll movement of the one or more IDMs may be achieved through the IDM mount flange 601. The robotic arm may position a robotic endoscope to an initial position (e.g., access point, natural orifice) to access the target tissue. In some embodiments, the robot arm can be passively moved by an operator. In such case, an operator can push the arm in any position and the arm compliantly moves (e.g., gravity compensation, admittance control, adaptive control, etc.). As shown in FIG. 4, in some embodiments, the robotic end effector of the robotic arm may comprise user interface feature 450 for an operator to position the IDM to a desired location. The handle may allow for gross positioning of the IDMs relative to the patient. In some cases, the user interface feature 450 may comprise a handle and one or more sensors (passive or active) that communicate to the controller of the robotic system when a user intends to position or reposition the robotic end effector. The robot arm can also be controlled in a compliant mode to improve human robot interaction. For example, the compliant motion control of the robot arm may employ a collision avoidance strategy and the position-force control may be designed to save unnecessary energy consumption while reducing impact of possible collisions. The arm may have redundant degrees of freedom allowing for its elbow to be algorithmically, or passively, moved into configurations that are convenient for an operator.

As described above, the robotic support system 400 may support one or more IDMs for driving one or more robotic instruments and the robotic endoscope. For example, a proximal end 1407 of a flexible robotic instrument (e.g., suturing instrument) may comprise a mechanical interface to allow the suturing instrument to be releasably coupled to an instrument driving mechanism as an end effector of a robotic support or a hand-held controller. The mechanical interfaces may comprise a set of actuators for communicating mechanical energy between the IDM and the robotic instrument for the steering or positioning of the end effector of the instrument end effectors within the surgical site.

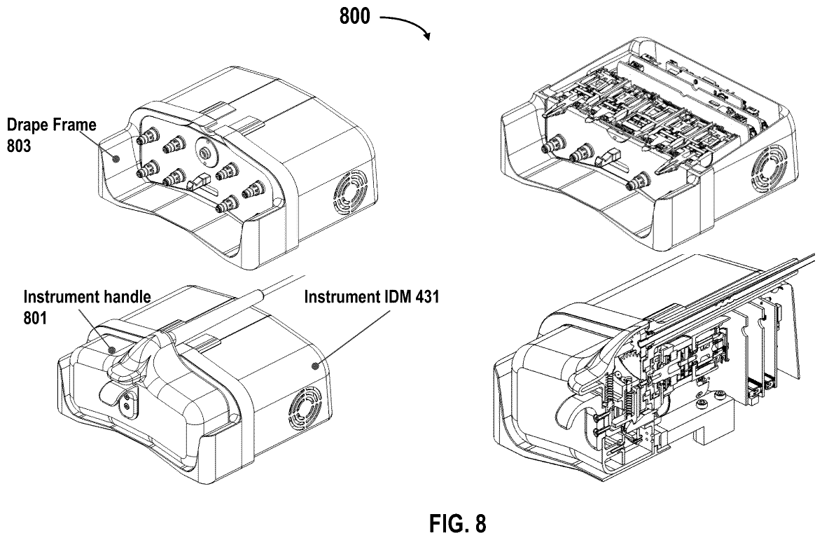

FIGS. 5, 7, and 8 show examples of one or more instrument driving mechanisms (IDM) for one or more robotic instruments. The instrument driving mechanism (IDM) 431, 433 for robotic instruments may comprise a set of motors 535 that are actuated to rotationally drive a set of pull wires of the elongate member of the robotic instrument to control an articulation of the bending section (e.g., bending section 1403 in FIG. 14), as well as controlling an operation of the end effector (1401 in FIG. 14) such as the needle driver operations. The proximal end 1407 of a robotic instrument 1400 may be mounted onto the instrument drive mechanism 431 or 433 so that its pulley/capstans assemblies are driven by the set of motors 535. FIG. 8 shows another example of a handle portion of a robotic instrument 801 releasably coupled to an instrument IDM 431. The number of pulleys may vary based on the pull wire configurations and/or the end effector operation. In some cases, one, two, three, four, or more pull wires may be utilized for articulating the flexible robotic instrument (e.g., bending section articulation in two or more degree of freedoms) and for driving the motion of the needle end effector. In some cases, one pull wire may be coupled to and driven by a pulley. In some cases, more than one wires may be coupled to a driven pulley. For example, two or more wires may be coupled to the same driven pulley antagonistically to drive the needle end effector motion such that rotation of the pulley provides tension to one wire(s) while slacking the other(s).

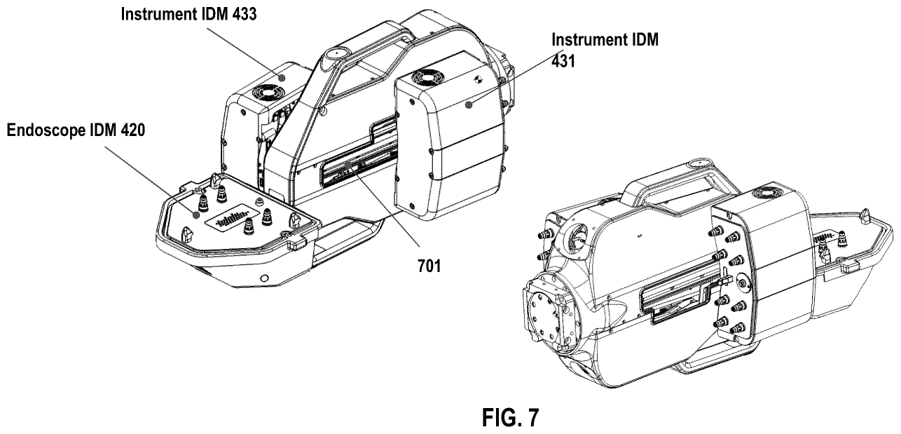

The mechanical actuator interfaces across the more IDMs for driving one or more robotic instruments and the robotic endoscope may be of the same type (e.g., uniform interface such as same set of motors, same geometries). Alternatively, the mechanical actuator interfaces across the more IDMs for driving one or more robotic instruments and the robotic endoscope may have different geometries, to different set of motors. As shown in FIG. 7, the IDM 431, 433 for the robotic instrument may have a geometry and a number of motors different from the geometry and motors of the IDM for the endoscope 420. The IDM mechanical interfaces may be different depending on the system requirements including loads and displacements required at each actuator interface.

In some embodiments, the one or more instrument IDMs 431, 433 may be able to translate 701 relative to the endoscope IDM 420. Such translational motion may beneficially facilitate a robotic instrument insertion and retraction through the robotic endoscope (e.g., robotic endoscope attached to the endoscope IDM 420).

In some embodiments, the robotic endoscope system may comprise a drape providing a sterile barrier that covers the robotic end effector 907 (e.g., IDMs) and the robotic arm 410 to maintain a sterile barrier as necessary for given surgical procedures. The drape (e.g., drape 440 in FIG. 4) may include interfaces for aligning with, coupling to and facilitating exchange with the robotic scope and robotic instruments. As shown in FIG. 8, the one or more IDMs may have mechanical interfaces 803 for locating and securing the drape and instrument handles 801. These interfaces may beneficially resist the loads applied between the IDM and instruments while ensuring the electrical connections remain reliable throughout use.

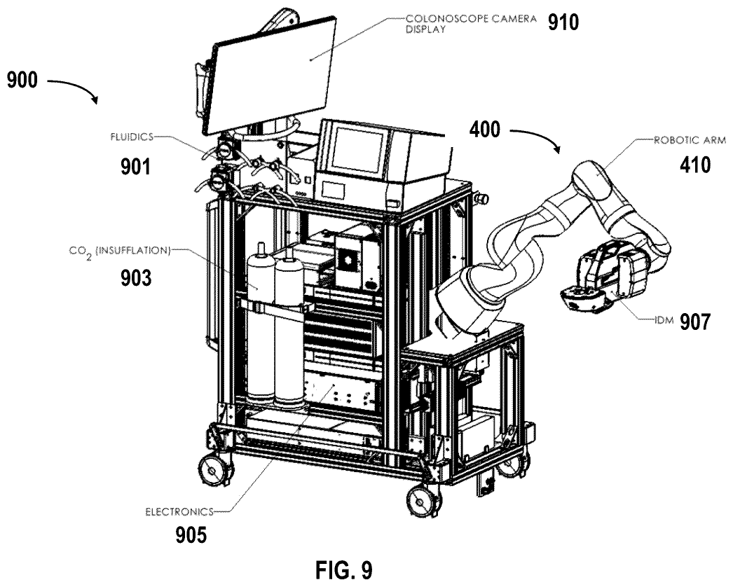

In some cases, the robotic end effector and robotic arm (e.g., 907 and 410 in FIG. 9) may be draped prior to coupling the robotic endoscope and robotic instruments to the robotic end effector. For example, to prepare the robotic cart 900 and surgeon console 910, the robotic end effector 907 and robotic arm 410 on the robotic cart may be draped and be positioned relative to the patient and the operating room to facilitate a smooth transition to the robotic colonoscope.

FIG. 9 shows an example of a treatment control system or a robotic cart 900. In some embodiments, the treatment control system 900 may include or be integrated with a robotic support system 400 including the robotic arm 410, instrument driving mechanism 907, robotic control unit 905, and one or more peripheral equipment such as irrigation and aspiration systems 901, 903 for managing fluidics, insufflation and smoke evacuation and various other functions. For example, the carbon dioxide (CO2) insufflation 903 may comprise an insufflator that is pressure-controlled to control flow of CO2 gas to inflate a space, such as the abdomen or digestive tract, for a variety of medical procedures. The insufflator 903 may be connected to insufflation channels and sensor channels that run through the robotic endoscope for the insufflation process. For example, abdominal Insufflation (Pneumoperitoneum) laparoscopic surgery begins with intraabdominal placement of the robotic endoscope comprising the insufflation feature, followed by carbon dioxide (CO2) insufflation of the abdominal cavity to an intraabdominal pressure (IAP) of 12 to 15 mm Hg. It should be noted other types of gas (e.g., air) may be utilized depending on the medical procedure.

The mobile cart 900 may include various elements 905 such as rechargeable power supply in electrical communication with an electric panel providing charging ports for portable electronic devices, converters, transformers and surge protectors for a plurality of AC and DC receptacles as power source for the on-board equipment including one or more computers storing application specific software for the treatment interface module.

The robotic endoscope system herein may comprise a user interface and/or a user console for visualizing the surgical site (e.g., camera view provided by the endoscope camera), controlling delivery of electrosurgical capabilities (e.g., via foot pedals) and user interface devices (handles and positioning systems) for capturing surgeon inputs for robotic control. The user console or user input device may provide a user interface for a user to configure the console to the clinician including adjusting the position and orientation of the viewer, configuring the user input devices and arm rest relative to one another and globally. FIG. 9 shows an example of a user interface 910 for visualizing the camera view or image/video captured by the colonoscope in real time. The user interface may be rendered at a display device 910 mounted to the robotic cart 900. The display may or may not be a touchscreen. The display may be a light-emitting diode (LED) screen, organic light-emitting diode (OLED) screen, liquid crystal display (LCD) screen, plasma screen, or any other type of screen. The display may be configured to show a user interface (UI) or a graphical user interface (GUI) rendered through a software application (e.g., via an application programming interface (API) executed on the system). In some cases, the live camera view may be overlaid with virtual renderings, e.g., augmented reality information.

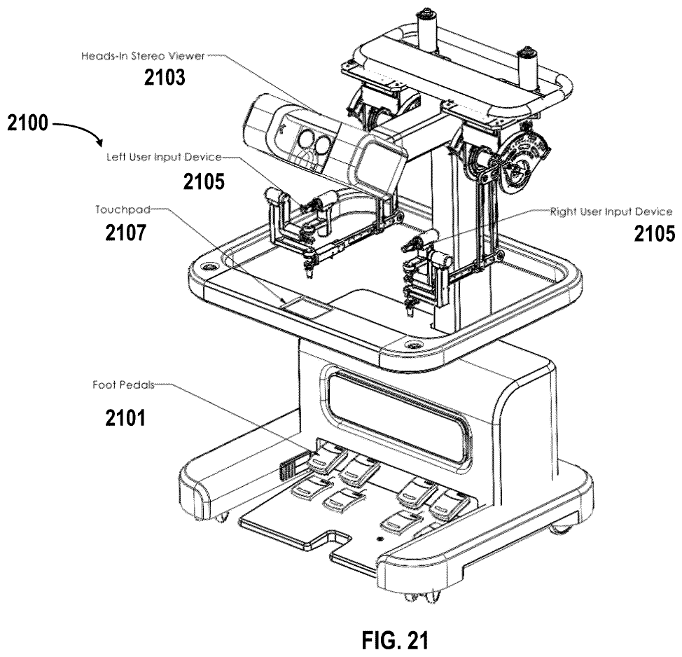

FIG. 21 shows an example of a user console 2100. The user console may allow an operator or user to interact with the colonoscope remotely during surgical procedures. The user console may comprise one or more user input device such as foot pedals 2101 for a variety of controls including, but not limited to, electrosurgical energy delivery, needle firing (in the case of a purpose-build suturing device) and clutching (e.g., to reposition the robotic scope tip).

In some cases, the user console may comprise a treatment interface module 2103 configured to provide a user interface displaying information related to using of the colonoscope such as navigation information, user information (e.g., control parameters), robotic endoscope camera view, and the like. Users may view the live colonoscope information, or sensor data via any suitable user interface 2103 (e.g., a display, heads-in stereo viewer, immersive, virtual reality (VR) and augmented reality (AR), etc.). The user console or a component of the user console (e.g., treatment interface module) may be mounted to the robotic cart 900. Alternatively or in addition to, the user console or a component of the user console (e.g., treatment interface module) may be mounted to a separate mobile cart or a remote system.

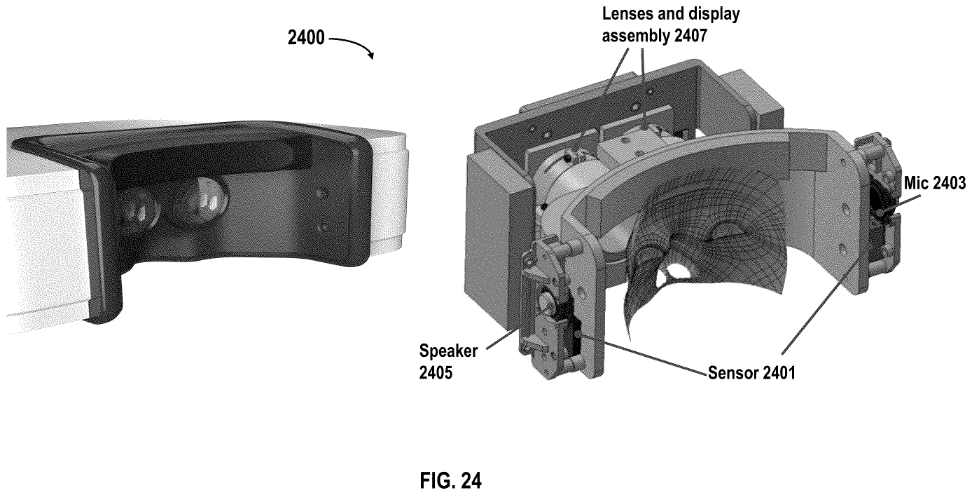

FIG. 24 shows an example of a stereo viewer apparatus 2400. The stereo viewer apparatus 2400 can be the same as the heads-in stereo viewer 2103 described in FIG. 21. In some embodiments, the stereo viewer apparatus 2400 may comprise a head-in immersive visualization unit to generate, transmit and display colonoscope camera data (e.g., live camera view) with GUI overlays. As shown in the illustrated example, the stereo viewer apparatus may comprise one or more sensors 2401 such as such as a proximity sensor to detect a presence of head. For instance, when the sensor detects presence of an operator's head is in place, the display may be activated, otherwise the display ma, otherwise the display ma be deactivated. In some cases, the stereoscopic imaging system may comprise at least two image sensors or lenses and assembly 2407 for displaying the stereoscopic view. The stereo viewer apparatus may also comprise other components such as Mic 2403 and speaker 2405 for the user to interact with the robotic platform (e.g., receiving audible feedback and providing voice command).

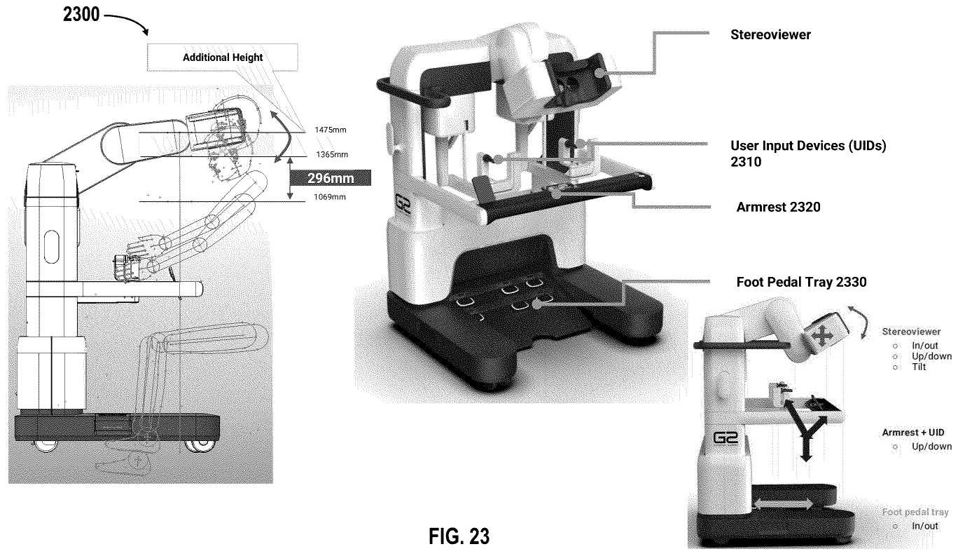

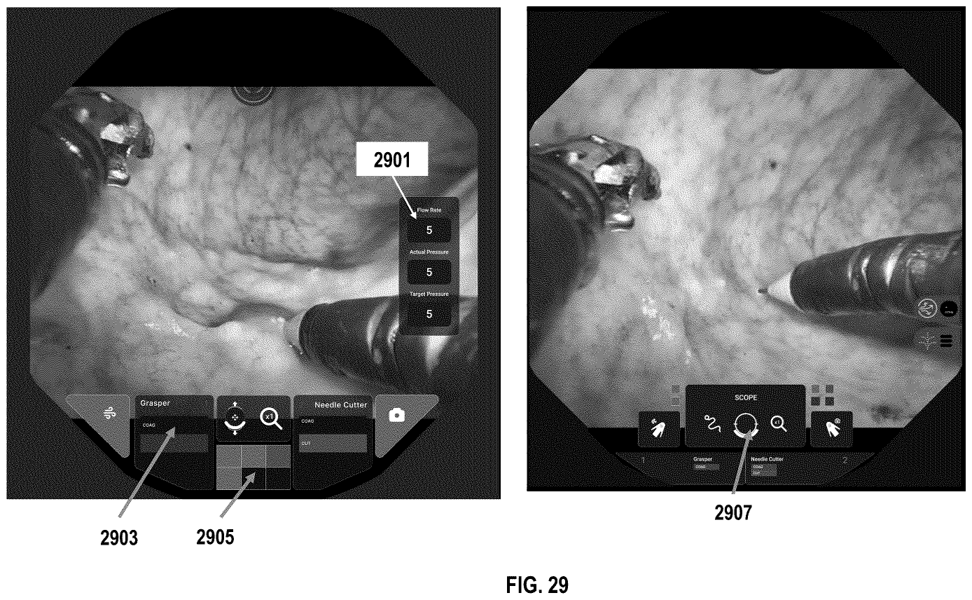

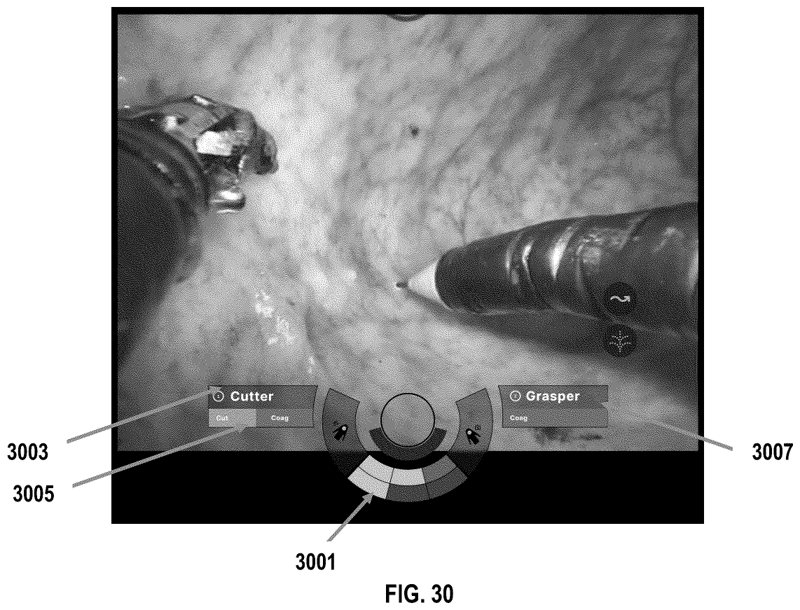

In some cases, the GUI overlays may comprise texts or graphical elements corresponding to pedal presses, instrument information, and other user operational information and/or system information. FIGS. 29 and 30 show examples of the display of the stereo viewer apparatus. In the illustrated example, the GUI overlays displayed on top of the live camera view may comprise status of one or more tools such as gasper 2903, cutter 3003, status 3005 (e.g., cut or Coag), grasper 3007 and its status. The GUI overlays may also comprise operational information 2901 such as the follow rate, actual pressure, target pressure and the like. In some cases, the GUI overlays may further comprise graphical elements corresponding to the foot pedals and pedal presses 2905, 3001. For example, the graphical elements corresponding to the foot pedals that are pressed or the foot is hovering over may be displayed with a color indicator. This beneficially allows a user to be informed of the position of the foot pedal without leaving the head-in stereo viewer. In some embodiments, the GUI overlays may further comprise information about the instrument. For example, an orientation of the instrument or scope 2907 may be displayed to provide the roll angle information. Referring to FIG. 23, the foot pedals or the foot pedal tray 2330 may comprise a hover sensor positioned above each pedal. The sensor may detect a foot hovering over the pedal before pressing on it. In some cases, upon detection of a foot or object hovering over a pedal, the corresponding GUI overlay may be displayed with a color indicator informing the user about the foot position and the presence of the foot relative to the pedal. As shown in the example, in some embodiments, the foot pedal tray may have a tiered structure. For example, the plurality of pedals may be arranged into multiple rows each at a different height. The difference in height may create a clearance so that a press on a top pedal will not actuate a bottom pedal. The hover sensor may e positioned right above each pedal to detect presence of a foot. The hover sensor may be able to detect a top pedal hovering presence without creating a false positive detection on the bottom pedal. For example, the detection may be based on an algorithm that when a presence of object is detected on both the top and bottom pedal, the algorithm may determine the foot is hovering over the top pedal. In some embodiments, the functions corresponding to each pedal may be re-mappable or configurable based on user preference. For example, the system may provide a user interface allowing a user to drag-drop a function to a selected pedal to customize the pedal functions and position of pedals.

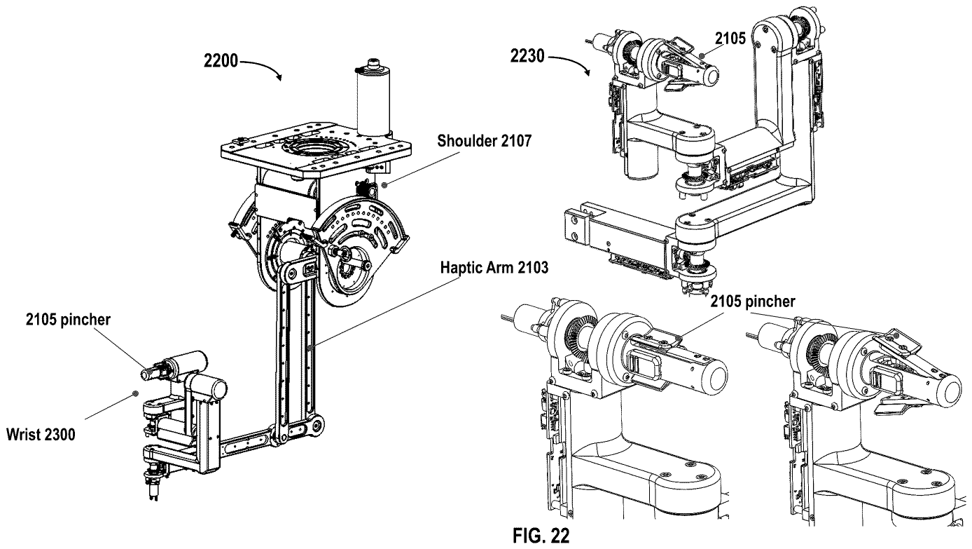

Referring back to FIG. 21, in some cases, the user console may comprise one or more user input devices such as touchscreen monitors, touchpad 2107, joysticks, keyboards and other interactive devices 2105 such as shown in the example of FIGS. 21 and 22. In some embodiments, a user may be able to navigate and/or control the motion of the robotic arm and the movement of the robotic endoscope using a user input device 2105. In some embodiments, the user console may comprise a left user input device 2105 and a right user input device 2105 corresponding to the left hand and right hand movement.

In some cases, the user input device may be in the form of an ‘arm’ comprising a shoulder assembly 2101, a wrist assembly 2230 and a pincher 2105 for controlling the robotic instrument. FIG. 22 shows examples of user input devices 2200. The user input device may be utilized to control the robotic endoscope and/or the robotic instrument such as by capturing and translating the user's hand input into an instrument output.

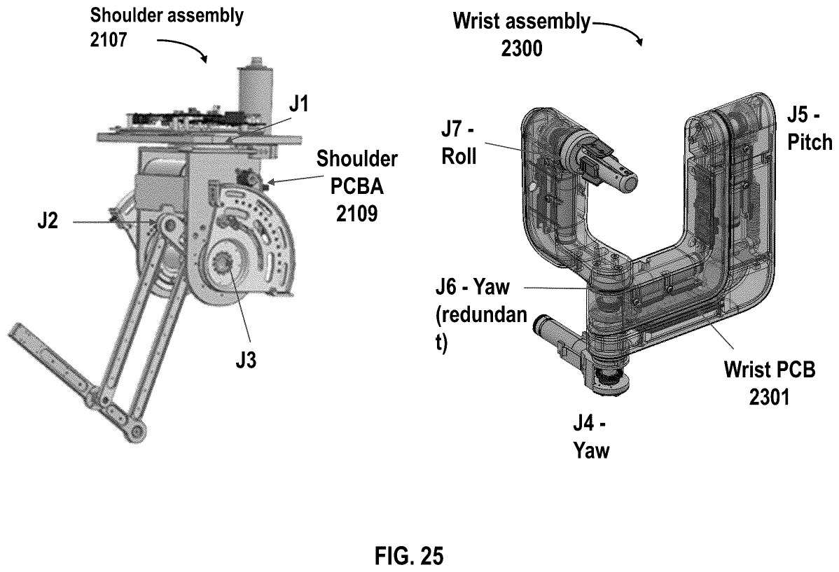

In some embodiments, each arm may comprise a series of links, joints actuated by motors for capturing a user's hand movement in at least six degrees of freedom. For instance, the shoulder assembly 2107 may comprise a haptic arm 2103 for controlling translation movement. In some embodiments, the wrist assembly 2230 may control a distal orientation. In some embodiments, the pincher assembly 2105 may control an end effector movement of the instrument such as jaw movement and rotation. The haptic arm 2103 and wrist assembly 2230 may comprise a series of links and joints allowing for at least six degrees of freedom. In some cases, each arm may comprise 5, 6, 7, 8, or more motors. For example, an arm may comprise seven motors. FIG. 25 shows an example of seven joints (J1-J7) of each arm actuated by seven motors. In some cases, the plurality of joints may have redundant axis (e.g., J6 redundant yaw axis) to provide additional flexibility or range of motion.

In some cases, the wrist assembly 2230 may comprise a precision grip handle for the clinician to grasp. The handle may comprise one or more gripper paddles 2105 which are used for controlling an operation of the instrument end effector. In some cases, a user's input may be intuitively mapped to an operation of an end effector. For example, a user may use the one or more gripper paddles or pincher 2105 to open and close the jaws of an instrument in an intuitive manner. For instance, a pinch motion captured by the pincher 2105 may be mapped to the close motion of the jaws, and release of the pinch motion may be mapped to the open of the jaws. In some cases, the handle may comprise other input features such as a sliding button which may be used for a variety of purposes including, but not limited to, clutching, parking, swapping instruments, deploying or stowing instruments within the surgical field.

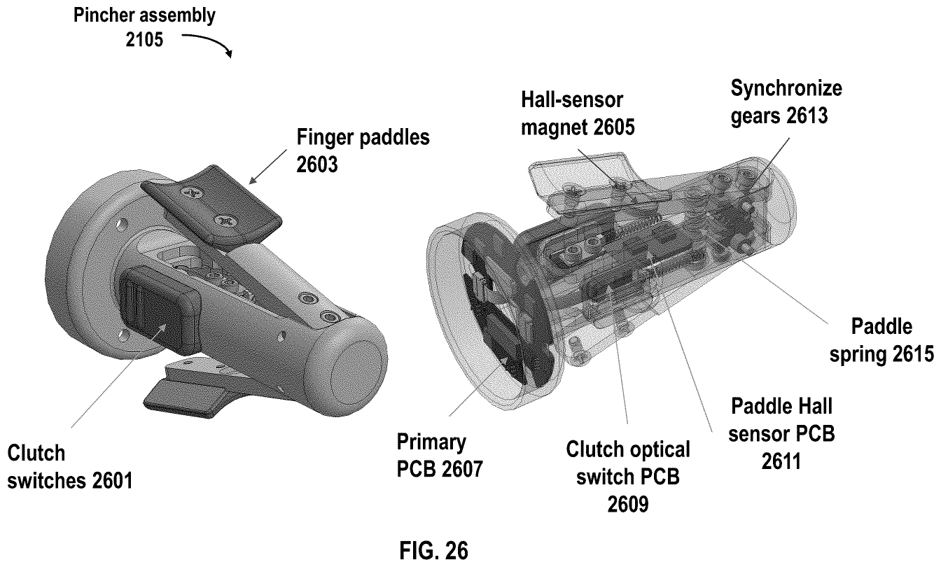

In some embodiments, each arm assembly may comprise a plurality of unique printed circuit board assemblies (PCBAs). As shown in FIGS. 25 and 26, the shoulder assembly may comprise a shoulder PCBA 2109 for controlling the motors actuating the joints of the shoulder assembly and converting the motion of the joints (e.g., measured by encoders) to signals mapped to the motion of the instrument (e.g., translational movement). Utilizing the PCBA can beneficially reduce Electromagnetic interference (EMI) which is unwanted noise or interference in the electrical circuit or path that can be caused by an electromagnetic field (EMF) from an outside source as well as cabling. In some embodiments, a PCBA 2109 of the shoulder assembly, or a PCBA 2301 of the wrist assembly may comprise a temperature and homing sensor, and motor controller. The temperature is used for thermal control/management of the motor. For example, the PCBA may comprise a connector for coupling to cooling features (e.g., fan connectors) and the thermal sensor may be used to monitor the temperature. The homing sensor may be included to provide stable repeatability of a homing position of the joint thereby improving positional accuracy of the joints.

FIG. 26 shows an example of a pincher assembly 2105 and various components of the pincher assembly. In the illustrated example, the pincher assembly may comprise one or more PCBs. For example, the pincher assembly may comprise a primary PCB 2607, a paddle hall-sensor PCB 2611, a clutch optical switch PCB 2609, and one or more sensors (e.g., hall-sensor magnet 2605). The pincher angle of a paddle may be measured by the hall effect sensors 2605 mounted to the paddles 2603. The plurality of PCBs may receive pincher angle (captured by the hall-sensor magnet mounted to the finger paddles 2603) and convert it to control signals to control a movement of the end effector. For example, an angle for opening a jaw of the end effector may be mapped to the pincher angle. Utilizing hall effect sensor instead of other positional sensors may beneficially improve the accuracy and sensitivity of the angle measurement for a fine control of the end effector operation. The plurality of PCBs may also receive signals from the button or clutch switches 2601 to disable the control action. The clutch switches 2601 or buttons may be activated when the movement of the wrist/shoulder assembly is beyond a range of motion. For example, when an operator moves the pincher to a position that is not comfortable, the operator may activate the clutch mechanism by pressing the clutch switches 2601 to decouple the arm assembly from movement of the instrument while moving the pincher to any comfortable position, and release the clutch switches to return to the control mode. Including the clutch switches on the sides of the pincher assembly allows users to conveniently control both the clutch function and paddles with a single hand thereby improving user experience of the control console.

In some embodiments, the pincher assembly 2105 may further comprise mechanical elements such as synchronize gears 2613 and paddle spring 2615 for capturing a pinching motion of a user.

As mentioned above, each arm assembly of the user console or user input device may comprise a plurality of motors or actuators. The motors or actuators may be calibrated for improving accuracy across different assembly variability. In some cases, the calibration may be conducted to identify a characterization relationship between a motor torque constant and different assembly variability.

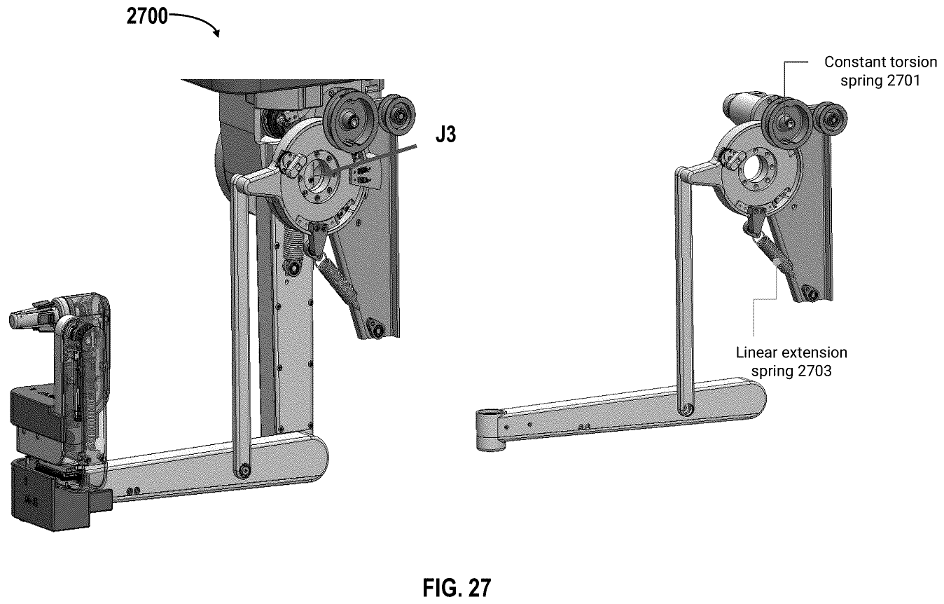

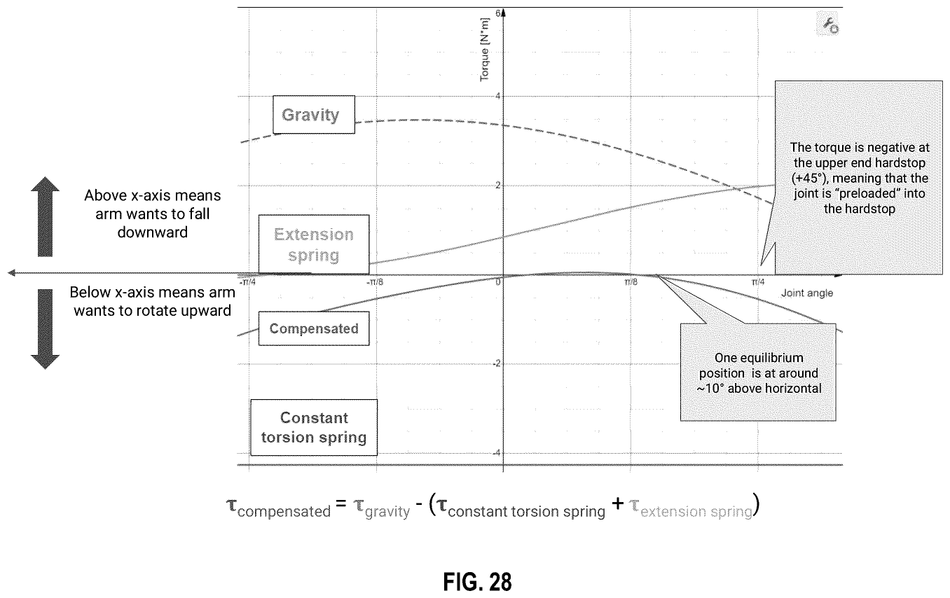

In some embodiments, the user input device may have passive compensation mechanism. The passive compensation mechanism may provide a safe stow position minimizing the risk of the user input device being damaged upon power off or during console transport. FIGS. 27 and 28 show examples of the passive compensation feature of the user input device 2700. As an example, at a stable stow position the wrist may be tilted forward and upward. In some cases, the passive compensation may comprise providing springs to joint 3 (J3) of the shoulder assembly. For example, constant tortional spring 2701 and linear extension spring 2703 may be included to provide the passive compensation to the joint 3. The springs may beneficially restrict a maximum torque applied to the joint by the weight of the assembly. FIG. 28 shows an example of the joint 3 passive compensation result. As shown in the example, the joint is compensated for the torque generated by the gravity reduced by the constant torsion spring and extension spring.

In some embodiments, the user console may have an ergonomic design accommodating different user preferences. Referring to FIG. 23, the user console 2300 may be integrated with a 6-axis ergonomic controller allowing for a tilt, translational movement of the stereo viewer, an up/down movement of the armrest 2320, and the in/out horizontal translational movement of the foot pedal tray 2330. In some cases, a GUI may be provided for a user to adjust or customize the ergonomic controls. In some embodiments, the stereo viewer positions may be expanded by the ergonomic design to allow for upright viewing posture. As shown in the example of the user console 2300, the range of motion may be expanded to increase the motion range for the up/down height adjustment, range of depth adjustment for the in/out of the stereo viewer, and range of tilt angle. The expanded motion range beneficially allows for flexibility for users of different heights or sitting postures to customize the user console based on their preferences.

In some cases, the user input device may include any other suitable input devices such as a tactile stylus device being physically in contact with a touch-sensitive display screen and the user may control the robotic system by moving the tactile stylus device on the display screen.

The user input device can have any type of user interactive component, such as a button, mouse, joystick, trackball, touchpad, pen, image capturing device, motion capture device, microphone, touchscreen, hand-held wrist gimbals, exoskeletal gloves, or other user interaction system such as virtual reality systems, augmented reality systems and the like. In some embodiments, the user may be permitted to personalize the user input device based on the personal preferences of the user such as handedness or the speed of driving the user interface device (e.g. the speed of moving a lever on a joystick for driving a robotic elongate member forward or backward). Artificial intelligence methods such as machine learning or deep learning may be used to personalize a user interface device based on user behavior. As an example, a machine learning method may be used to learn based on the user behaviors such as the use of buttons, use of levers, the frequency of used of buttons or levers, the number of clicks or the speed of moving the levers on a joystick to adapt and become specialized. For example, the user interface may be adapted to use a combination of buttons or levers for a specific task based on user preference for using those buttons and levers.

### Overtube

Certain endoscopes such as colonoscopes or gastroscopes may have relatively greater size and/or stiffness (compared to other types of endoscopes), making navigating tortuous anatomy can more challenging. For instance, clinicians may use an overtube to ease intubation thereby providing a low friction surface and a defined trajectory to guide the intubation of the colonoscope or gastroscope. In some cases, reduction method may be adopted to ease the intubation by reducing the length and tortuosity of the colon by anchoring to the colon at the distal end and applying tension. However, it can be challenging to use overtubes for intubation. One such challenge in the current colon intubation process is the large size of the overtube device (i.e., balloon overtube) used for making passage for the inner endoscope and assisting in intubation. For instance, for diagnostic and therapeutic procedures in bodily lumen, clinicians have traditionally intubated with manual endoscopes. In the colon, manual colonoscopy is able to successfully reach the target such as the cecum (the end of the large intestine) relatively easily (e.g., in less than 10 minutes) for most patients. However, the intubation process can be more challenging for robotic colonoscope system due to the increased size/dimension of the robotic colonoscope device. During an assisted or autonomous intubation process with robotic colonoscopes, overtubes may be utilized to ease the intubation. An overtube is a sleeve-like equipment usually made of semi-rigid plastic or silicone rubber, designed to assist endoscopy. An overtube of a larger diameter than that of an endoscope is needed for providing a route through the gastrointestinal (GI) tract.

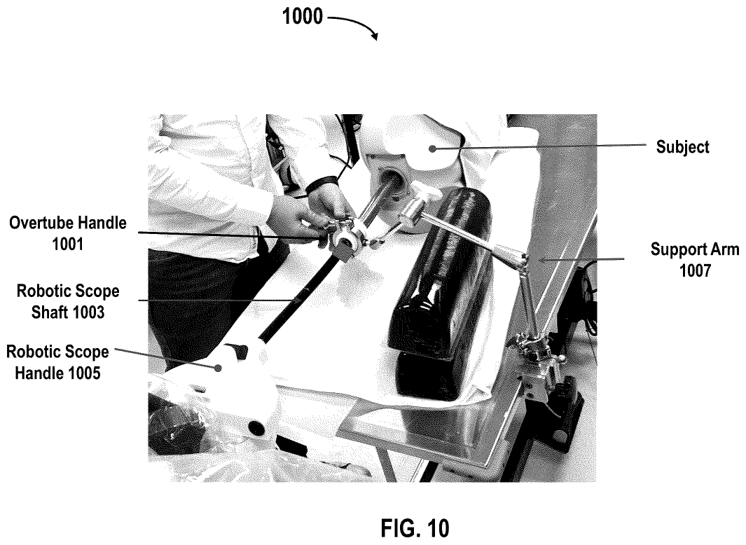

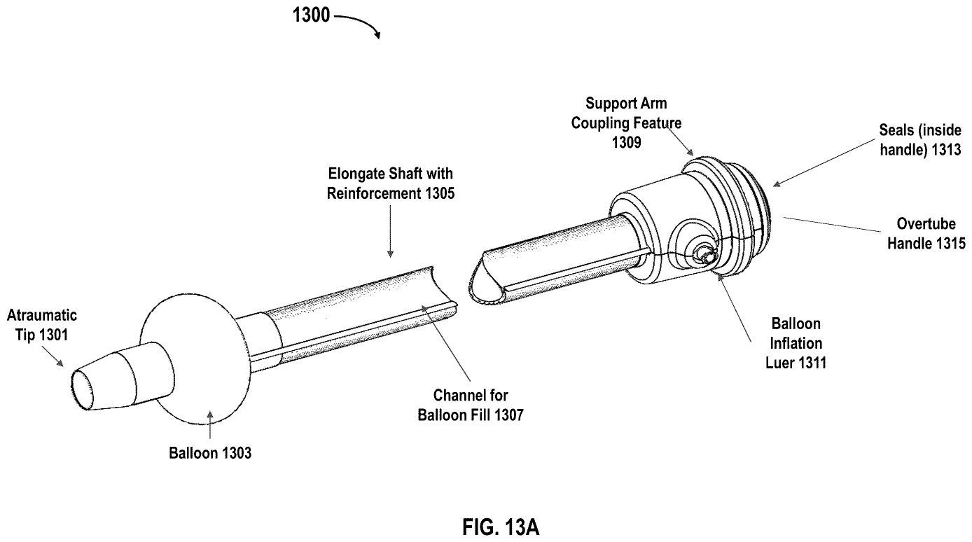

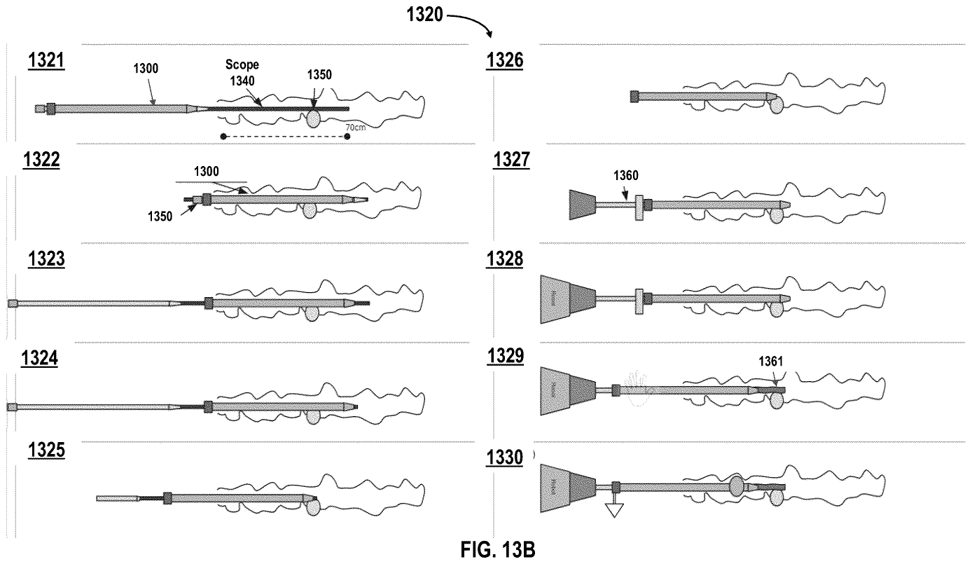

The present disclosure provides an improved overtube that is to be placed into the patient in order to gain access to the anatomical region to be examined and to provide a controlled conduit for accommodating the robotic endoscopes and the robotic instruments. The overtube may help with reduction (reducing the length of the colon and straightening it by means of anchoring to the colon wall and pulling tension on the tether to pull the anchored point on the colon towards the rectum). FIG. 10 shows an example 1000 of an overtube used for reducting an anatomy and facilitating scope exchange (e.g., exchange between a standard scope and a robotic scope of the present disclosure). The seals in the handle portion 1001 of the overtube beneficially prevents fluid and air leaks between the handle and the colonoscope shaft. For example, in some embodiments as illustrated in FIG. 13A, the exemplary overtube 1300 may comprise an overtube handle 1315 having seals 1313 instead the handle portion to prevent fluid and air leaks. The handle portion 1315 may have suitable exterior shape and/or geometries to facilitate coupling with a support arm. For example, the support arm coupling feature 1309 may be coupled to a support arm 1007 as shown in FIG. 10 to support the exposed robotic colonoscope shaft 1003 during an intubation procedure. The support arm 1007 can be any suitable articulated arm that is mounted to a patient table or a bed-side robotic cart allowing for the end effector to be movable in at least three degrees of freedom.

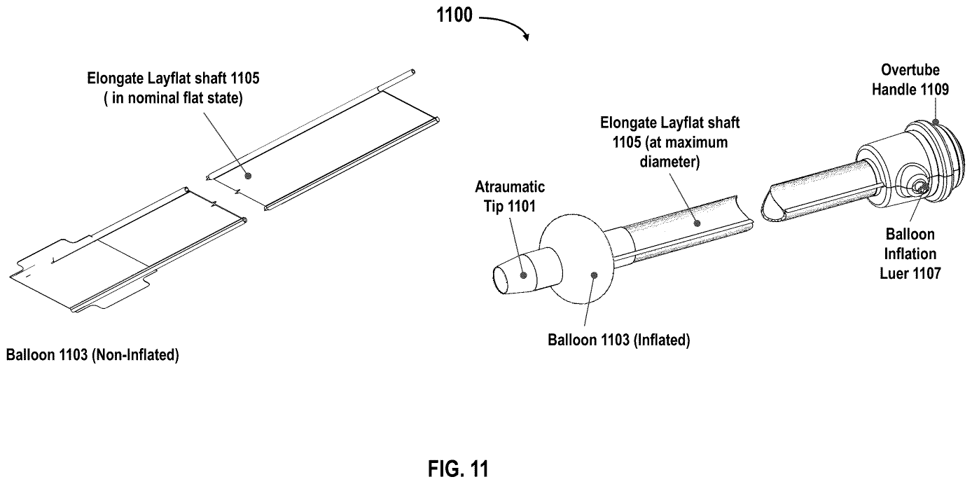

FIGS. 11 and 13A show examples of overtube. In the illustrated examples, the overtube 1100, 1300 may have an atraumatic tip 1101, 1301 to assist navigating in the tortuous anatomy. The overtube may comprise a lumen construction that retains its shape naturally (e.g. an elongate cylinder) or it may take on the shape dictated by the internal and external constraints (e.g. a lay-flat film construction). FIG. 11 illustrates an overtube device 1100 having a layflat tube construction. The inner diameter of the elongate layflat shaft 1105 is large enough to pass a robotic scope (e.g., gastroscope). The wall section of the overtube may comprise one or more films and may be segmented into multiple lumens. For instance, the overtube shaft may comprise at least one through channel (primary channel) and one or more auxiliary channels. For example, a resting state of the layflat shaft 1105 may be rolled along its longitudinal axis such that the roll of the one or more films forms a temporary lumen through which a first scope (e.g., standard colonoscope) can be inserted through.

In some cases, the one or more auxiliary channels may or may not be through channel. The one or more auxiliary channels may be populated with other tools or constructions and can be used for a variety of purposes. For example, two auxiliary channels may be formed by sealing the film along its primary axis where the two auxiliary channels are arranged on either side of the primary channel. The auxiliary channels may be populated with small polymer tubes to provide axial stiffness to facilitate the transmission of forces (tension and compression) along the axial axis direction between the overtube handle 1109 and the overtube tip 1101. In some cases, the small polymer tubes may also be utilized to provide a channel for fluid to be delivered from the handle to the balloon 1103 which is attached at the distal tip.

FIG. 13A illustrates an overtube device 1300 having a tubular construction. In some embodiments, the wall section of the elongate shaft 1305 may be reinforced by additional components such as a coil, braid or others. The reinforcement components may be used to maintain the cross-section of the tube when the inner diameter is not in use and to facilitate the transmission of forces and torques applied at the overtube handle 1315 for advancing and rotating the overtube. Alternatively, the overtube shaft may not have reinforcement components. In some embodiments, the overtube may have an inner diameter greater than the outer diameter of the robotic colonoscope such that an interstitial space between the overtube and the colonoscope may be for smoke evacuation. During an initial intubation, the overtube may be coupled to a standard scope (e.g., a colonoscope with smaller diameter or a manual scope) and the assembled standard scope and overtube may be advanced together through the colon to the target site. When the target site is reached, the overtube balloon 1303 may be inflated. The balloon inflation may allow the overtube to anchor to the colon wall and the overtube can be pulled proximally to reduct (e.g., shorten and straighten) the colon. The standard scope (e.g., smaller colonoscope) may be removed from the colon and a robotic scope (e.g., Gastroscope) may be placed through the primary lumen of the overtube up to the target site. The inflation and deflation of the balloon 1303 may be controlled by connecting the balloon inflation luer 1311 to fluidics and delivery the fluidics through the channel for balloon fill 1307. In some cases, the balloon may be inflated manually with a standard saline-filled syringe.

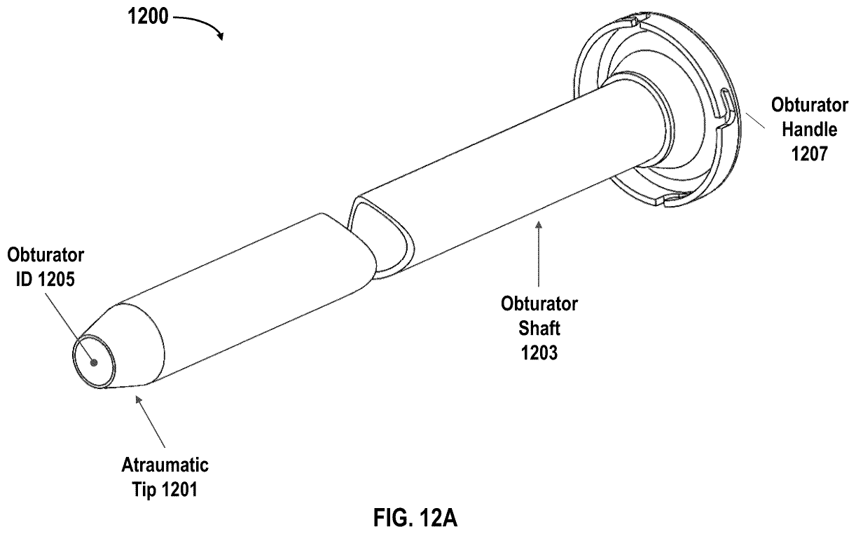

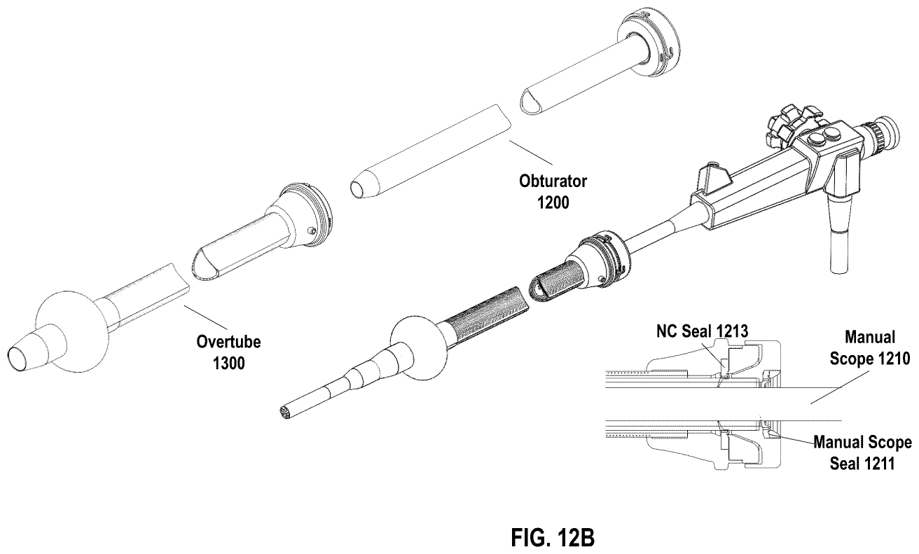

In some embodiments, the overtube may be used in combination with a dilator to occupy the space between the overtube inner diameter and the outer diameter of the smaller scope (e.g., smaller diameter colonoscope or standard scope) during an initial intubation. FIGS. 12A and 12B shows an example of a dilator/obturator 1200 used in conjunction with an overtube 1300. In some embodiments, the dilator or obturator shaft 1203 may be constructed of a wall section and may be treated with coatings on the inner and outer diameters to facilitate smooth translation with respect to the overtube 1300 and the colonoscope 1210. The wall section of the obturator shaft 1203 may be reinforced with additional reinforcement elements or structures. Alternatively, the wall section of the obturator shaft 1203 may be non-reinforced. As described above, the dilator may be utilized during an initial intubation where the overtube is coupled to a smaller colonoscope (e.g., manual scope 1210) and advanced together to a target site. The dilator may be used to occupy the space between the overtube 1300 inner diameter and the outer diameter of the smaller scope (e.g., manual scope 1210 or standard colonoscope). The dilator or obturator may eliminate the radial clearance between the smaller colonoscope outer diameter and the larger overtube inner diameter. In some embodiments, the smaller scope (e.g., manual scope 1210 or standard colonoscope) may comprise a seal 1211 at the proximal end and the obturator may comprise a seal 1213 at the proximal end.

In an aspect of the present disclosure, an improved intubation workflow is provided. The workflow may comprise coupling a robotic colonoscope to a robotic drive mechanism (instrument driving mechanism (IDM)) after the colonoscope has been intubated and with the colonoscope in a position and orientation dictated by the curvature of the patient's anatomy. The method may comprise intubating a robotic endoscope with an overtube, then coupling the intubated robotic endoscope to an IDM once it is inside a subject's body. The overtube may be the same as those described elsewhere herein.

FIG. 13B shows an exemplary intubation workflow 1320 for an endoscope device. The endoscope device may be a robotic endoscope device such as a robotic colonoscope or gastroscope as described elsewhere herein. The robotic endoscope device may have an increased dimension due to additional components for the robotic control or robotic features compared to a manual or standard colonoscope. For instance, the robotic endoscope may have a larger-than-normal diameter.

The intubation workflow 1320 may comprise intubating 1321 with a first scope 1340 (e.g., manual scope, manual colonoscope), placing an overtube 1300, and advancing the first scope 1340 until reaches a position that may be passed a target site 1350. The first scope 1340 can be any available endoscope that can be manually inserted into a patient lumen. The first scope may have a dimension smaller than a dimension of a second scope such as a robotic scope that is to be intubated. For example, a diameter of the scope 1340 may be smaller than a diameter of a robotic scope 1360. The overtube 1300 may be advanced over the scope 1340. The intubation step can be a convention process such as the bending section of the scope 1340 is advanced over the overtube's tip and then the overtube is advanced to the tip of the scope 1340 until they reach or pass over a target site 1350. The initial intubation process 1322 may comprise coupling an obturator/dilator 1350 to the overtube as described above. The overtube can be the same as those described above such as a coil reinforced overtube.

Once the scope tip reaches a position that past over the target site or site of interest 1350, the workflow may comprise an operation 1322 of advancing the overtube 1300 along the bending section of the scope 1340 to place a balloon proximal to the tip of the scope 1340. Next, the workflow may comprise an operation 1323 of inflating the balloon and removing the scope 1340 while leaving the overtube in place 1324. In some cases, the colon may be reducted 1325 such that the tip of the overtube may be located at the target site 1326 once the first scope is removed. The overtube is equipped with a distal balloon for anchoring to the patient anatomy. In some cases, the balloon may be inflated manually with a standard saline-filled syringe.

Next 1327, a second scope such as a robotic endoscope (e.g., gastroscope or robotic colonoscope) 1360 is inserted through the overtube 1300. In some cases, the robotic endoscope 1360 may be inserted manually. In some cases, the robotic colonoscope may be inserted into the overtube until the colonoscope tip is just shy of the overtube tip or does not extend over the overtube. In some cases, the insertion of the robotic endoscope into the overtube may be controlled by monitoring a relative position of the markings along the robotic scope shaft and the overtube handle. Once the robotic endoscope 1360 is placed at the site of interest, the proximal end or the handle of the robotic endoscope 1360 may be connected to a robotic drive mechanism or instrument driving mechanism (IDM) 1328. The workflow may proceed with an operation 1329 of deflating the balloon of the overtube and the overtube may be pulled in the proximal direction to expose the bending section 1361 of the robotic endoscope. In the next operation 1330, the balloon of the overtube is inflated and the overtube may be grounded such as via a grounding mechanism or support arm located at a proximal end of the overtube.

The robotic endoscope 1360 may then be capable of performing any controlled operations at the site of interest. In some cases, the robotic operations may comprise an improved initialization process provided by the present disclosure. Once the initialization process is completed, a user or operator may control the robotic colonoscope 1360 during a procedure. The colonoscope may be operated by a user robotically such as from a surgeon console which is positioned away from the patient. Once the procedures is completed, the robotic colonoscope is disconnected from the robotic drive mechanism and is removed along with the overtube from the patient. The overtube may be single-use or disposable. The robotic colonoscope 1360 may be single-user or disposable. Alternatively, at least part of the robotic colonoscope is reusable.

In some cases, operator may confirm the robotic colonoscope position and orientation on the bed-side cart monitor (e.g., 910 in FIG. 9). A user may control navigation of the robotic colonoscope using the user input devices (e.g., hand-held controller, gross positioning of the robotic end effector, etc.) and the user console may also permit the user to view the site of interest as described elsewhere herein.

In some cases, one or more robotic instruments such as grasper and cutter instruments may be inserted into the respective working channels of the robotic scope. For example, the robotic instruments may be inserted from the bed-side location and the instrument handles may be connected to the IDMs on the robotic end effector. FIGS. 14-20 show robotic instruments with various end effectors that can be utilized in the provided robotic endoscope system herein.

### Robotic Instruments

As described above, the robotic endoscope device may have a working channel allowing tools such as graspers, cutters or suturing instruments to pass through. In another example, a suturing device can be coupled to the distal end of an endoscope, which enables suturing in the gastroesophageal tract of a patient. The robotic endoscope may have a bending section and a rigid tip or end effector attached to the bending section. In order to be able to be maneuvered through bodily lumens, the dimension (length) of the digital tip portion of a robotic endoscope is desired to be as small as possible so that it can pass through tortious pathways. However, current instrument devices (e.g., suturing devices) are designed for manual endoscope devices such as laparoscopy which typically has a length or rigid structure greater than a desired dimension for an end effector. It is desirable to provide end effectors for instruments (e.g., suturing devices) suitable for robotic endoscopic platforms or used endoluminally.

FIG. 14 schematically shows an example of a robotic instrument 1400. The robotic instrument 1400 may comprise an end effector 1401 located at a distal end of an elongate member. The elongate member may be an articulatable, flexible member comprising a bending section 1403, a shaft 1405 and a proximal end 1407. The proximal end 1407 may be a handle that is releasably attached to IDM at a robotic end effector. In some cases, the proximal end 1407 may comprise driving components (e.g., pulley) that are releasably coupled to an instrument driving mechanism (IDM) to drive an operation of the instrument end effector 1401 (e.g., needle operation, ferrule retention mechanism operation, gasper operation, cutter operation, etc.) and/or the motion (e.g., articulation) of the bending section 1403.

In some cases, the proximal end 1407 may comprise a mechanical interface to allow the robotic instrument to be releasably coupled to an instrument driving mechanism attached to a robotic support or a hand-held controller. The instrument driving mechanism (IDM) can be the same as the IDM 431, 433 as described above. For example, the instrument IDM may comprise a set of motors 535 that are actuated to rotationally drive a set of pull wires of the elongate member to control an articulation of the bending section, as well as controlling an operation of the instrument end effector such as the needle driver motion, grasper operation, cutter operation, and the like. The proximal end 1407 may be mounted onto the instrument drive mechanism 431, 433 so that its pulley/capstans assemblies are driven by the set of motors 535. The number of pulleys may vary based on the pull wire configurations. In some cases, one, two, three, four, or more pull wires may be utilized for articulating the flexible robotic instrument (e.g., bending section articulation in two or more degrees of freedom) and for driving the motion of the instrument end effector. In some cases, one pull wire may be coupled to and driven by a pulley. In some cases, more than one wire may be coupled to a driven pulley. For example, two or more wires may be coupled to the same driven pulley antagonistically to drive the needle end effector motion such that rotation of the pulley provides tension to one wire(s) while slacking the other(s).

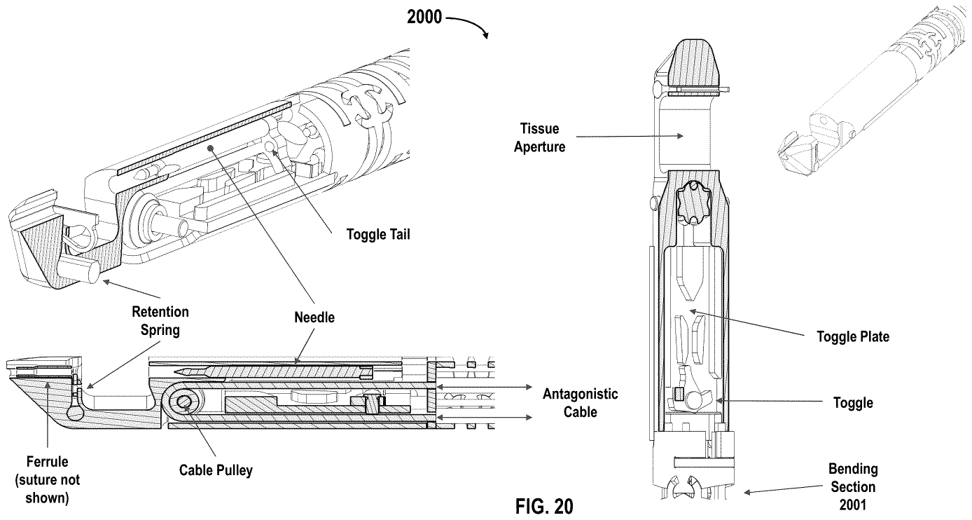

The bending section 1403 may be articulated in one or more degrees of freedom. The articulation of the bending section 1403 may be controlled by applying force to the distal tip portion of the elongate member via the one or multiple pull wires. A distal end of the one or more pull wires may be attached to the distal end of the robotic instrument 1400. In the case of multiple pull wires, pulling one wire at a time may change the orientation of the end effector 1401 to pitch up, down, left, right or any direction needed. In some cases, the pull wires may be anchored at the distal tip portion of the robotic instrument 1400, running through the bending section, and entering the proximal end they are coupled to a driving component (e.g., pulley). This pulley may interact with an output shaft (e.g., output shaft 537 in FIG. 5) from the robotic system. In some cases, one or more of the pull wires may be utilized for the end effector drive mechanism (e.g., needle driver in FIG. 20) to drive a translational or displacement movement of the needle (e.g., drive forward and backward motion of the needle in the end effector).

In some embodiments, the proximal end or portion of one or more pull wires may be operatively coupled to various mechanisms (e.g., gears, pulleys, capstans, etc.) in the proximal end. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the robotic instrument, such that operation of the pull wires by the control unit may apply force or tension to the distal portion which may steer or articulate (e.g., up, down, pitch, yaw, or any direction in-between) at least the distal portion (e.g., bending section) of the suturing instrument. The pull wires may be made of any suitable material such as stainless steel (e.g., SS316), metals, alloys, polymers, nylons or biocompatible material. In some embodiments, different pull wires may be made of different materials for varying the load bearing capabilities of the pull wires.

The shaft 1405 may connect the bending section 1403 to the proximal handle 1407. The shaft 1405 may be flexible and a passive section. For example, the active bending section 1403 may be designed to allow for bending in two or more degrees of freedom (e.g., articulation). A greater bending degree such as 180 and 270 degrees (or other articulation parameters for clinical indications) can be achieved by the unique structure of the active bending section 1403. In some cases, the active bending section 1403 and the passive section or shaft 1405 may be fabricated separately as a modular component and assembled to the elongate member. In some cases, the cut patterns of the active bending and shaft may be different such that at least the minimum bend radius of the two sections may be different. In some cases, a variable minimum bend radius along the axial axis of the 1405 member may be provided such that an active bending section 1403 or the passive shaft section 1405 may comprise two or more different minimum bend radii. In some cases, the active bending section 1403 and the shaft 1405 of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius).

The end effector 1401 may be attached to the bending section. The end effector may be robotically controlled to perform a variety of surgical tasks including, but not limited to, the following: an end effector configured with a thin cannulated tip for injecting fluid such as lifting agent, where the cannulated tip may also have monopolar capability for cauterizing tissue at the command of the clinician; an end effector configured with a grasping functionality where at least one jaw is movable with respect to the other; an end effector configured with a dissecting functionality with the tapered and pointed distal tip of the jaws; an end effector for wound closure, including an interface specially designed for managing the insertion and retrieval of a needle and suture through tissue; an end effector for the installation of surgical clips, anchors or cinch-type devices to retain tissue into clinician-desired folds or appositions; and an end effector with scissors or shears for cold cutting tissue or surgical materials such as suture and the like.

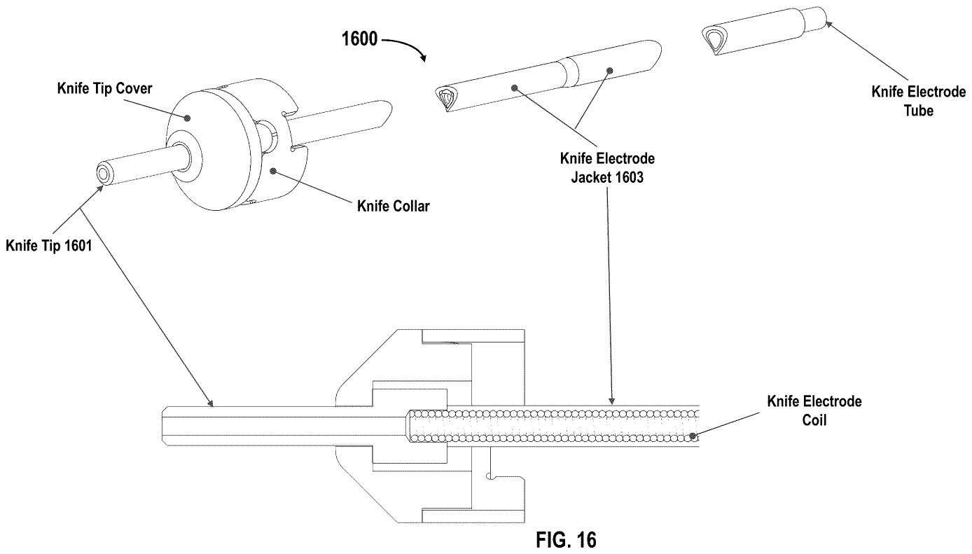

FIGS. 16-20 show some examples of the end effectors for robotic instruments. In some embodiments, the robotic instrument may include a robotic cutter instrument. FIG. 16 shows an example of an end effector for a robotic cutter 1600 in accordance with some embodiments of the present disclosure. In some cases, the robotic cutter instrument may be an electrosurgical instrument that is capable of delivering fluids to the surgical site. The cutter tip 1601 of the robotic cutter instrument contains a lumen for fluid delivery and the tip stem is electrically coupled to an electrosurgical generator through circuitry in the instrument shaft and instrument handle. The body of the cutter may be electrically insulating (e.g., electrode jacket 1603) to prevent electrosurgical energy from coupling to tissue that is not in contact with the stem. The cutter tip 1601 may be independently steerable (e.g., controlled via the instrument IDM) from the robotic scope such that the instrument shaft can be advanced and retracted, and rotated relative to the robotic scope. In some cases, the distal bending section of the instrument shaft may be articulated for changing the angle of the instrument tip with respect to the tissue.

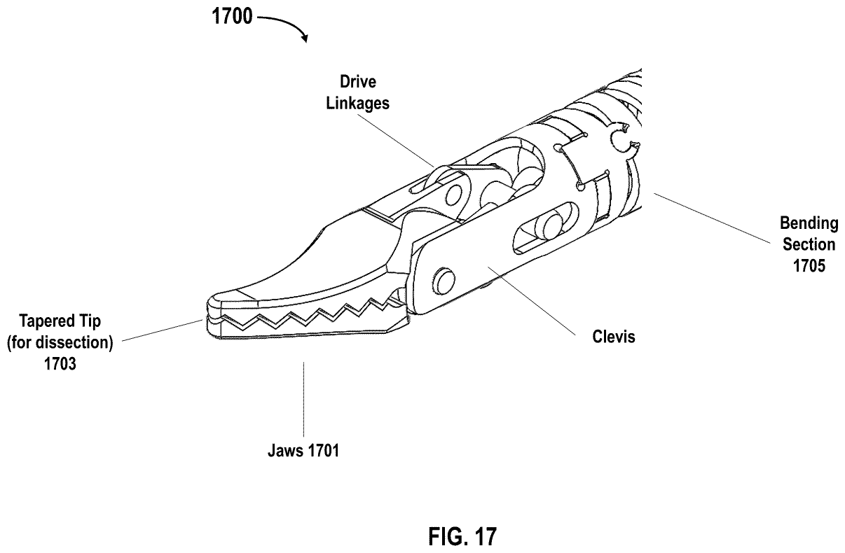

The robotic instrument may include a robotic gasper instrument. FIG. 17 shows an example of an end effector for a robotic grasper 1700 in accordance with some embodiments of the present disclosure. The grasper may have a pair of jaws 1701 with fine teeth for grasping and manipulating tissue. In some embodiments, the grasper may comprise a tapered tip 1703 for aiding with dissection. The grasper is independently steerable (e.g., controlled via the instrument IDM) from the robotic scope such that the instrument shaft can be advanced and retracted, and rotated relative to the robotic scope. In some cases, the distal bending section 1705 of the instrument shaft may be articulated for changing the angle of the instrument tip with respect to the tissue.

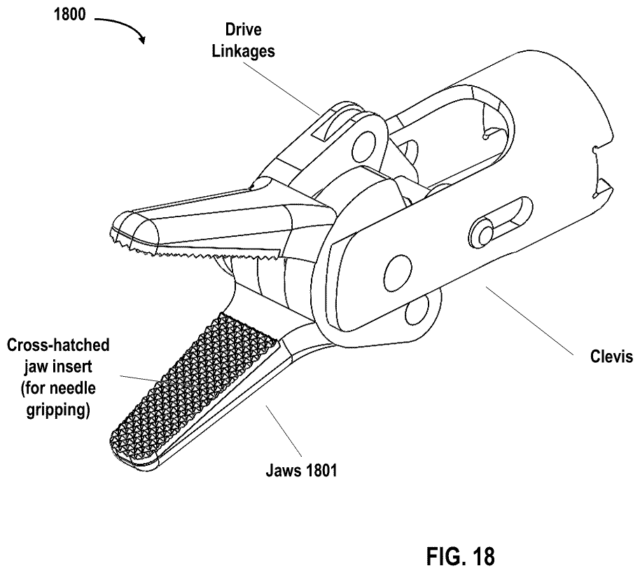

The robotic instrument may include a needle driver instrument. FIG. 18 shows an example of a robotic needle driver instrument 1800 in accordance with some embodiments of the present disclosure. The needle driver 1800 may be a grasper-like instrument for suturing with a standard needle and thread. The needle driver may have a pair of jaws 1801 with fine, cross-hatched teeth for gripping small needles. The needle driver is independently steerable (e.g., controlled via the instrument IDM) from the robotic scope such that the instrument shaft can be advanced and retracted, and rotated relative to the robotic scope. In some cases, the distal bending section of the instrument shaft may be articulated for changing the angle of the instrument tip with respect to the tissue.

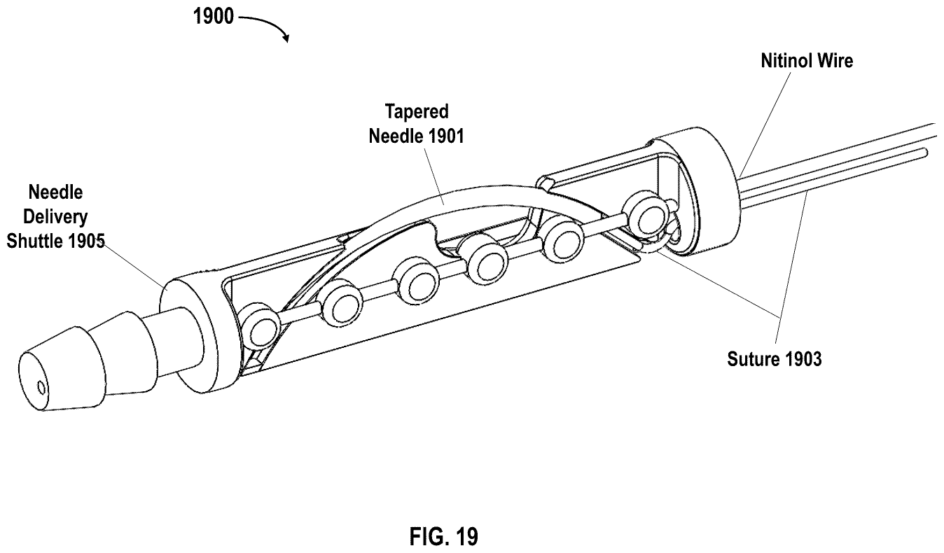

FIG. 19 shows an example 1900 of an end effector for a needle delivery tool. In some embodiments, the robotic instrument may be purpose-built for delivering a curved needle 1901 through a working channel to the surgical site. Depending on the needle geometry, the robotic instrument may be inserted through the auxiliary channel (e.g., smaller diameter) or the working channels (e.g., larger diameter). The needle delivery tool 1900 may package the curved needle 1901 such that the plane of curvature is aligned with the axis of the instrument for safely delivering through the working channel and into the surgical site without exposing the needle tip. In some cases, suture 1903 is packaged within the shaft of the needle driver tool. Once in the surgical site, the needle may be removed from the needle delivery tool. In some cases, removal of the needle may be performed by grasping the needle using one of the robotic instruments and removing the needle from the insertion needle delivery shuttle 1905. In some cases, needle removal may be performed by employing a needle ejection mechanism within the needle delivery tool 1900. For example, the needle delivery tool may comprise a mechanism operated within the instrument shaft, moving a wedge relative to the needle and forcing the needle from a retained state to discharge the needle into the surgical field. The needle can then be picked up with one of the other instruments. The needle delivery tool end effector may be actuated with a manual handle, a robotic handle or a combination of both.

FIG. 20 shows an example 2000 of an end effector for a robotic suturing instrument. The suturing device instrument may allow suturing with a custom needle and thread. Unlike a traditional needle driver, which has opposing jaws for grasping onto a free needle, the suturing device shown in the figures has a captive needle that is separable from the suture thread. The needle is straight and aligned with the axis of the instrument. When actuated, the needle crosses the aperture in the end effector from proximal to distal, penetrating the tissue and then coupling to the ferrule which is attached to the suture. When retracted, the needle and ferrule are both drawn through the tissue. The tissue is removed from the instrument aperture and the needle and ferrule are cycled again to deposit the ferrule and suture into the distal end of the end effector, resetting for another pass through tissue. This robotic suturing instrument may be used for suture and needle introduction into the surgical field, eliminating the need for a separate needle delivery instrument.

The end effector 2000 may have two (e.g., roll and translation), three (e.g., roll and articulation), four (e.g., roll, articulation and translation) or more degrees of freedom. For example, the end effector may have a roll movement (e.g., rotatable about the longitudinal axis of the elongate member), articulatable about two axes (e.g., via the articulation of the bending section). The end effector may also have translational movement (e.g., insertion and retraction of the needle). In some cases, the roll movement of the end effector may be achieved via a wrist at the distal portion of the suturing instrument such that the end effector 2000 may have a roll movement relative to the elongate member of the suturing instrument. Alternatively, the end effector 2000 may not have a roll movement relative to the bending section 2001. In some cases, the roll movement of the end effector may be achieved via the roll movement of the elongate member, a wrist located at the distal end of the bending section 2001 or a combination of both.

The robotic endoscope platform herein is capable of performing a variety of complex endoluminal surgical operations or surgical procedures, including, but are not limited to, endoscopic submucosal dissection (ESD), endoscopic mucosal resection (EMR), Endoscopic Gastric Plication, full thickness resection and the like.

FIG. 15 shows an example of performing an endoscopic submucosal dissection (ESD) using the robotic endoscopic platform herein. In an exemplary process, a patient is positioned on an operating room table and is prepared for the procedure. The bed-side cart (e.g., robotic mobile cart 900 in FIG. 19) and surgeon console is prepared. For instance, preparing the bed-side cart may comprise draping the robotic end effector (e.g., 907 in FIG. 19) and robotic arm (e.g., 410 in FIG. 19) on the bed-side cart and pre-positioning the robotic system (e.g., 400 in FIG. 19) relative to the patient and the operating room to facilitate a smooth transition to the robotic colonoscope. Alternatively, the bed-side cart may be draped and positioned out of the way such that the drape remains “clean” (or sterile) and the bed-side cart does not interfere with the initial intubation. In some cases, the robotic system may not be prepared until the site of interest is identified and targeted, to minimize the costs of not using the system in the event that the lesion identified is metastasized according to a lesion classification system and clinician expertise.

Next step is intubation. The intubation process can be similar to those described elsewhere herein. For example, an overtube may be prepared for use by removing from the packaging and activating the hydrophilic surface coatings as necessary and as desired. The overtube may be assembled with a standard scope (e.g., diagnostic colonoscope). In the case of colonoscope, lubrication may be applied to the patient's anus and the colonoscope tip as necessary. The diagnostic colonoscope is inserted into the patient's anus and is navigated to the site of interest (e.g., operation 1321 in FIG. 13B). During the insertion, video and images may be acquired or recorded. The overtube and the standard scope may be used to reduct the colon to facilitate intubation. The intubation process can be the same as those described in FIG. 13B. For instance, the standard scope is replaced with the robotic colonoscope. Once the robotic colonoscope is navigated to the target site (a user may navigate the robotic colonoscope tip and visualize the site of interest via the surgeon console), one or more robotic instruments may be inserted into the working channels of the robotic colonoscope.

As shown in FIG. 15, a user may deploy the robotic instruments from the colonoscope by controlling the robotic instruments via the surgeon console, under visualization. A user may control the robotic instruments via the user input device at the surgeon console. For example, a user may perform “Clutch-In” to control the robotic instruments by navigating the user input devices on the surgeon console to a comfortable starting position and orientation and opening/closing the grips of the user input device to match with the robotics instruments.

Next, a user may perform the lesion dissection by retracting and lifting with the grasper, while injecting lifting agent and cauterizing the tissue planes with the cutter to remove the lesion. Once the lesion has been removed from the colon wall, a user may remove the lesion from the colon entirely by holding the specimen with the robotic grasper or auxiliary grasper while removing the robotic scope and robotic instruments from the surgical site. Alternatively, a user may remove the lesion by placing a Basket into the working channel to capture and retain the specimen while removing it from the surgical site. In some cases, the robotic arm and end effector may be retracted enough to completely withdraw the robotic scope and specimen from the anatomy without undocking the robotic colonoscope from the robotic arm.

The system allows for camera visualization to be maintained throughout the procedure, including during the insertion of tools through the working channel and the entire surgical process performed by the robotic instrument. For example, as described above, the exit ports 301 at the distal tip of the robotic scope may allow the robotic grasper 1700 and cutter 1600 to have triangulation by making the arms spread out or divert away from the base as they exit the distal portion of the primary sheath distal portions and then be steered back toward each other and utilized to apply capturing and/or compressive loads to a subject tissue structure, and the like, with the field of view of the image capture device 313 preferably capturing such activity from any desired location relative to the robotic instruments (e.g., grasper, cutter).

The tissue sample such as the lesion may be measured off site. In the case of diagnosis process, the tissue sample may be rapidly evaluated on-site by a rapid on-site evaluation process to determine whether repetition of the tissue sampling is needed, or to decide further action. In some cases, the rapid on-site evaluation process may also provide a quick diagnostic analysis on the tissue sample to determine the following surgical treatment. For instance, if the tissue sample is determined to be malignant as a result of the rapid on-site evaluation process, the robotic treatment instruments (e.g., grasper, cutter) may be inserted through the working channel of the robotic colonoscope to remove the lesion. This beneficially allows for diagnosis and treatment being performed in one session.

Next, a closure device (e.g., a needle driver 1800, 1900 or a suturing device 2000) may be inserted through the working channel or auxiliary channel of the robotic colonoscope to perform surgical wound closure. The robotic instrument handle may be docked to the instruments IDMs at the robotic end effector. If needed, the grasper instrument may be re-inserted and the instrument handle is docked to the robotic end effector. Next, the robotic instruments may be deployed from the robotic colonoscope tip via control at the surgeon console to perform the suturing operations. If a needle driver is used (e.g., needle driver 1800), a needle deliver instrument (e.g., 1900 in FIG. 19) may be introduced to deliver the needle via the auxiliary port. If a closure device is used, the suture tail is retrieved from the working channel with the grasper instrument. A user may use the grasper and other instrument to close the wound with the needle and suture as necessary and as desired.

Once the closure operation is completed, a manual scissor may be inserted through the auxiliary channel of the robotic colonoscope to cut the needle from the suture thread. A user may remove the needle under direct camera visualization in a similar process as tissue/specimen retrieval.

A user may confirm homeostasis of the wound via the camera view and confirm the effective diameter of the residual colon at the site of the closure to provide confidence that the closure does not significantly restrict the diameter of the colon. Upon confirmation, a user may retrieve the robotic instruments and robotic colonoscope from the anatomy, and retrieve the overtube from the anatomy. In some cases, disinfection, sterilization or reprocessing may be performed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for performing a robotic endoscopic procedure, the method comprising:
(a) inserting a robotic endoscope into a bodily lumen of a subject using an overtube as a guide, wherein the robotic endoscope is mechanically decoupled from a robotic drive mechanism during said inserting;
(b) advancing the robotic endoscope through the overtube until a distal end of the robotic endoscope is positioned at a target site within the bodily lumen;
(c) after the advancing, coupling a proximal end of the robotic endoscope to an instrument driving mechanism (IDM) of a robotic support system while the distal end of the robotic endoscope remains at the target site inside the subject; and
(d) after the coupling, robotically controlling the robotic endoscope via the IDM from a user console.

2. The method of claim 1, wherein (a) further comprises: performing an initial intubation by advancing a first scope and the overtube together through the bodily lumen to the target site, wherein the first scope has an outer diameter smaller than an outer diameter of the robotic endoscope, anchoring the overtube at or proximal to the target site by inflating a distal balloon of the overtube, and withdrawing the first scope from the overtube while the balloon remains inflated, leaving the overtube anchored in the bodily lumen.

3. The method of claim 2, wherein the initial intubation further comprises coupling a dilator to the overtube such that the dilator occupies an interstitial space between an inner surface of the overtube and an outer surface of the first scope, thereby reducing radial clearance between the overtube and the first scope during the advancing.

4. The method of claim 2, further comprising, after (c), deflating the balloon of the overtube and retracting the overtube in a proximal direction to expose an articulatable bending section of the robotic endoscope and re-inflating the balloon of the overtube to anchor the overtube relative to the bodily lumen.

5. The method of claim 1, wherein the inserting of the robotic endoscope through the overtube further comprises monitoring a position of the robotic endoscope within the overtube by tracking relative positions of markings along a shaft of the robotic endoscope with respect to a handle of the overtube.

6. The method of claim 1, wherein the bodily lumen is a colon, and wherein the advancing further comprises reducing the colon by anchoring the overtube to a wall of the colon via the distal balloon and applying proximal tension to the overtube to shorten and straighten the colon.

7. The method of claim 1, wherein the overtube comprises an elongate shaft having a wall section reinforced by at least one of a coil or a braid, thereby maintaining a cross-sectional shape of the overtube and facilitating transmission of axial forces and torques applied at a handle of the overtube for advancing and rotating the overtube within the bodily lumen.

8. The method of claim 7, wherein the overtube further comprises a seal within the handle of the overtube that prevents fluid and air leaks between the handle and a shaft of the robotic endoscope passing therethrough.

9. The method of claim 1, further comprising evacuating surgical smoke through an interstitial space defined between an outer surface of a shaft of the robotic endoscope and an inner surface of the overtube, and wherein an inner diameter of the overtube is greater than an outer diameter of the shaft of the robotic endoscope.

10. The method of claim 1, further comprising inserting one or more robotic instruments through respective working channels of the robotic endoscope and coupling one or more proximal ends of the one or more robotic instruments to one or more instrument IDMs on a robotic end effector of the robotic support system.

11. The method of claim 10, further comprising activating a clutch mechanism to decouple the one or more instrument IDMs from controlling a movement of the one or more robotic instruments.

12. The method of claim 11, wherein the user console comprises a stereo viewer apparatus configured to display a stereoscopic real-time camera view from the robotic endoscope to a user.

13. The method of claim 12, wherein the stereo viewer apparatus is adjustable in at least height, depth, and tilt to permit an upright viewing posture, and wherein the method further comprises adjusting the stereo viewer apparatus to a position selected by the user based on the user's height or sitting posture.

14. The method of claim 13, wherein the stereo viewer apparatus comprises a proximity sensor configured to detect a presence of the user's head at the stereo viewer apparatus, and wherein the method further comprises activating a display of the stereo viewer apparatus upon detecting the presence and deactivating the display upon non-detection.

15. The method of claim 14, further comprising displaying, overlaid on the real-time camera view in the stereo viewer apparatus, one or more graphical overlay elements comprising at least one of: a status information for one or more robotic instruments, an operational information, or a graphical indicator corresponding to a foot pedal of the user console.

16. The method of claim 15, further comprising detecting, via a foot-presence sensor positioned above a foot pedal of the user console, that a user's foot is hovering above the foot pedal without pressing the foot pedal; and displaying, in response to the detecting, an indicator in the graphical overlay element corresponding to the foot pedal to inform the user of the foot's position relative to the foot pedal.

17. The method of claim 16, wherein the user console comprises a tiered foot pedal tray comprising a plurality of foot pedals arranged in at least two rows at different heights, and wherein the detecting further comprises: receiving simultaneous detection signals from foot-presence sensors positioned above a first foot pedal in an upper row and a second foot pedal in a lower row, and determining, based on the simultaneous detection, that the user's foot is hovering over the first foot pedal in the upper row.

18. The method of claim 1, wherein the user console comprises a user input device including a shoulder assembly, a wrist assembly, and a pincher assembly.

19. The method of claim 18, wherein the user input device comprises a passive compensation mechanism that applies a restoring force to at least one joint of the shoulder assembly to counteract a torque generated by gravity acting on the user input device, thereby maintaining the user input device in a stable stow position when robotic control is not active.

20. The method of claim 18, wherein the pincher assembly comprises one or more hall-effect sensors configured to measure a pincher angle of one or more gripper paddles and to generate control signals corresponding to a degree of opening or closing of an end effector of a robotic instrument based on the pincher angle.
